(19) United States
(12) Patent Application Publication
(10) Pub. No.: US 2023/0204582 A1
(43) Pub. Date: Jun. 29, 2023

(54) CYTOPATHOLOGICAL STAINING

(71) Applicant: CYTOBAY INC., Santa Monica, CA (US)

(72) Inventors: Wenjiang Chu, Santa Monica, CA (US); Jian Wang, Santa Monica, CA (US)

(21) Appl. No.: 17/999,542

(22) PCT Filed: May 21, 2021
(86) PCT No.: PCT/US2021/033672

§ 371 Date: Nov. 21, 2022

Related U.S. Application Data
(60) Provisional application No. 63/091,633, filed on Oct. 14, 2020.

(30) Foreign Application Priority Data
May 28, 2020 (CN) 202010467313.3
May 28, 2020 (CN) 202010468262.6
Oct. 10, 2020 (WO) PCT/CN2020/120070
Oct. 10, 2020 (WO) PCT/CN2020/120071

(51) Int. Cl. G01N 33/574; G01N 1/30

(52) U.S. Cl. CPC G01N 33/57407; G01N 1/30; G01N 2474/00

(57) ABSTRACT

Disclosed herein are in vitro methods of multiple staining and slide preparation for a cytopathological sample, such as a urine sample. These methods enable simultaneous staining of biomarkers and cytopathological stains to greatly enhance confidence in identifying atypical cells such as cancer cells. Also disclosed herein are methods of using said staining and preparation methods for the detection of bladder cancer using urine samples from a patient. These methods offer increased sensitivity and specificity over conventional bladder cancer detection methods. Also disclosed herein are the urinary exfoliated cells stained according to the methods provided herein.

### CROSS-REFERENCE TO RELATED APPLICATIONS

This application claims the benefit of priority to U.S. Provisional Pat. Application No. 63/091,633, filed Oct. 14, 2020, International Pat. Application PCT/CN2020/120071, filed Oct. 10, 2020, designating the United States and published in the English language, which claims the benefit of priority to Chinese Application 202010468262.6, filed May 28, 2020, and International Patent Application PCT/CN2020/120070, filed Oct. 10, 2020, designating the United States and published in the English language, which claims the benefit of priority to Chinese Application 202010467313.3, filed May 28, 2020, each of which is hereby expressly incorporated by reference in its entirety.

### FIELD OF THE INVENTION

Aspects of the present disclosure relate generally to multiple staining and slide preparation methods in the field of cytopathology. These methods can be used for the detection of bladder cancer in urine samples.

### BACKGROUND

Bladder cancer, the fourth most common cancer in men and the twelfth most common cancer in women, is estimated to account for 81,400 new cancer cases and 17,980 deaths in the USA in 2019. There are approximately 829,620 bladder cancer survivors in the USA in 2019.

Urine cytology is highly effective for the detection of high-grade and high-stage bladder cancer. However, it proves relatively ineffective as a tool to detect low-grade malignancy with reported sensitivity for detection of low-grade tumors ranging only 4% to 31%. This is because cytopathologic features of low-grade urothelial carcinoma morphologically resemble those of normal bladder urothelial cells. It is exceedingly difficult to diagnose low-grade urothelial carcinoma based on cytopathologic features in urine sediment samples. Furthermore, false positive rates can be as high as 12% due to inflammation, urothelial atypia, and changes caused by chemotherapy and radiation.

These limitations hinder urine cytology utilization and indicate that significant improvement in urine cytology is manifest. There is a lasting need for new and better cytological methods for identifying and detecting urothelial carcinoma and other bladder cancers in urine samples obtained non-invasively.

### SUMMARY

Disclosed herein are methods for the in vitro cytopathologic detection of atypical cells using a combination of diagnostic cytomorphology with chromogenic immunocytochemistry and/or in situ hybridization staining of patient derived cells. Performing both stains allows for observation of both expression of one or more cancer specific biomarkers and cytomorphological characterization by multiply staining the same urinary sediment cells. These staining methods are amenable for analysis under conventional light microscopes, eliminating the need for potentially costly or prohibitive equipment such as fluorescent microscopes. Biomarker staining can be done with protein-based or nucleic acid-based methods, such as immunocytochemistry and/or chromogenic in-situ hybridization. These multiple staining methods are used for the detection of bladder cancer cells in urine. In particular, the methods provided herein significantly improve positive evaluation of low-grade bladder cancer from urinary exfoliated cells, which are found in very little quantities in urine samples and must be conserved as much as possible. Also disclosed herein are the urinary sediment cells stained according to any of the methods provided herein.

Embodiments of the present disclosure provided herein are described by way of the following numbered alternatives:

1. A method of detecting bladder cancer in a patient, comprising:
isolating urinary exfoliated cells from the patient;
fixing the urinary exfoliated cells in an aqueous cell suspension;
staining the urinary exfoliated cells with a liquid-based immunocytochemistry or chromogenic in-situ hybridization stain, or both;
staining the urinary exfoliated cells with a diagnostic cytomorphologic stain;
assessing the urinary exfoliated cells as comprising bladder cancer cells by the immunocytochemistry or chromogenic in-situ hybridization stain, or both, and the diagnostic cytomorphologic stain;
thereby detecting bladder cancer in the patient.

2. The method of alternative 1, wherein the urinary exfoliated cells are urinary sediment cells isolated from the urine sample by centrifugation.

3. The method of any one of the preceding alternatives, wherein the immunocytochemistry or chromogenic in-situ hybridization stain, or both, is performed in an aqueous cell suspension.

4. The method of alternative 3, wherein the immunocytochemistry or chromogenic in-situ hybridization stain, or both, comprises labeling one or more bladder cancer specific biomarkers.

5. The method of alternative 4, wherein the one or more bladder cancer specific biomarkers are selected from the group consisting of S100P, p63, M344, LDQ10, 19A211, GATA-3, Ki-67, p16, Her-2, PD-L1, CTLA4, CK-17, CK-20, nmp-22, bladder tumor antigen (BTA), hTERT, and mini-chromosome maintenance protein 5 (MCM5).

6. The method of any one of the preceding alternatives, wherein the diagnostic cytomorphologic stain is performed either:
a) in an aqueous cell suspension; or
b) after mounting the urinary exfoliated cells onto a solid substrate.

7. The method of any one of the preceding alternatives, wherein the diagnostic cytomorphologic stain comprises Diff-Quik stain, Papanicolaou stain, Wright-Giemsa stain, hematoxylin/eosin stain, or a derivative or modification thereof.

8. The method of alternative 7, wherein the diagnostic cytomorphologic stain is free of or has a reduced concentration of an alcohol, such as methanol and/or ethanol.

9. The method of any one of alternatives 6-8, wherein the solid substrate is a microscope slide.

10. The method of any one of the preceding alternatives, wherein the assessing step is done by light microscopy, digital pathology slides, or artificial intelligence.

11. The method of any one of the preceding alternatives, wherein the assessing step includes counting the numbers of total bladder cancer cells, or percentage of biomarker positive cells among bladder cancer cells, or both.

12. The method of any one of the preceding alternatives, wherein the bladder cancer cells comprise one or more bladder cancer specific biomarkers selected from the group consisting of S100P, p63, M344, LDQ10, 19A211, GATA-3, Ki-67, p16, Her-2, PD-L1, CTLA4, CK-17, CK-20, nmp-22, bladder tumor antigen (BTA), hTERT, and mini-chromosome maintenance protein 5 (MCM5).

13. The method of any one of the preceding alternatives, wherein the bladder cancer is urothelial carcinoma, squamous cell carcinoma, adenocarcinoma, small cell carcinoma, sarcoma, or any combination thereof.

14. The method of any one of the preceding alternatives, wherein the bladder cancer is a low-grade bladder cancer.

15. The method of any one of the preceding alternatives, further comprising treating the patient for bladder cancer.

16. The method of alternative 15, wherein treating the patient for bladder cancer comprises surgery, chemotherapy, immunotherapy, targeted therapy, or radiation therapy, or any combination thereof.

17. The method of alternative 16, wherein the chemotherapy comprises administration of cisplatin, carboplatin, fluorouracil, mitomycin, gemcitabine, methotrexate, vinblastine, doxorubicin, erdafitinib, afatinib, docetaxel, or paclitaxel, or any combination thereof.

18. The method of alternative 16 or 17, wherein the immunotherapy comprises administration of Bacillus Calmette-Guerin (BCG), atezolizumab, avelumab, durvalumab, enfortumab, nivolumab, ipilimumab, trastuzumab, disitamab, PRS-343, or pembrolizumab, or any combination thereof.

19. The method of any one of the preceding alternatives, wherein the patient is a mammal.

20. The method of any one of the preceding alternatives, wherein the patient is a human.

21. The method of any one of the preceding alternatives, wherein the patient is selected to provide urinary exfoliated cells based on presentation of clinical symptoms of bladder cancer and/or being part of a population at high risk of developing bladder cancer.

22. The method of any one of the preceding alternatives, wherein any one or more of the antibodies or binding fragments thereof set forth in FIG. 3 are utilized.

23. The method of any one of the preceding alternatives, wherein assessing the urinary exfoliated cells by the diagnostic cytomorphologic stain comprises assessing characteristics of the urinary exfoliated cells comprising nuclear features, shape or size of the nuclei or nucleoli; the density of chromatin; cytoplasmic elements such as mucins, fat droplets or neurosecretory granules; or cell membrane features such as membrane grooves, projections, or vacuoles.

24. The method of any one of the preceding claims, wherein staining the urinary exfoliated cells with the liquid-based immunocytochemistry or chromogenic in-situ hybridization stain, and/or staining the urinary exfoliated cells with the diagnostic cytomorphologic stain comprises centrifugation or use of one or more inserts or transwells, wherein the one or more inserts or transwells comprise a porous membrane that permits aqueous solution to pass but retains the urinary exfoliated cells and, optionally, wherein the one or more inserts or transwells further comprise a flange, protrusion, tab, or handle.

25. A population of urinary exfoliated cells stained with a liquid-based immunocytochemistry or chromogenic in-situ hybridization stain, or both, and a diagnostic cytomorphologic stain, wherein the population of urinary exfoliated cells is isolated from a patient.

26. The population of urinary exfoliated cells of alternative 25, wherein the urinary exfoliated cells are urinary sediment cells isolated from a urine sample from the patient by centrifugation.

27. The population of urinary exfoliated cells of alternative 25 or 26, wherein the immunocytochemistry or chromogenic in-situ hybridization stain, or both, has been performed in an aqueous cell suspension.

28. The population of urinary exfoliated cells of any one of alternatives 25-27, wherein the population of urinary exfoliated cells is stained with the immunocytochemistry or chromogenic in-situ hybridization stain with one or more bladder cancer specific biomarkers.

29. The population of urinary exfoliated cells of alternative 28, wherein the one or more bladder cancer specific biomarkers are selected from the group consisting of S100P, p63, M344, LDQ10, 19A211, GATA-3, Ki-67, p16, Her-2, PD-L1, CTLA4, CK-17, CK-20, nmp-22, BTA, hTERT, and MCM5.

30. The population of urinary exfoliated cells of any one of alternatives 25-29, wherein the population of urinary exfoliated cells are stained for one or more bladder cancer specific biomarkers.

31. The population of urinary exfoliated cells of any one of alternatives 25-30, wherein the population of urinary exfoliated cells are stained for one or more of S100P, p63, M344, LDQ10, 19A211, GATA-3, Ki-67, p16, Her-2, PD-L1, CTLA4, CK-17, CK-20, nmp-22, BTA, hTERT, or MCM5

32. The population of urinary exfoliated cells of any one of alternatives 25-31, wherein the diagnostic cytomorphologic stain has been performed either:
a) in an aqueous cell suspension; or
b) after the population of urinary exfoliated cells have been mounted onto a solid substrate.

33. The population of urinary exfoliated cells of alternative 32, wherein the solid substrate is a microscope slide.

34. The population of urinary exfoliated cells of any one of alternatives 25-33, wherein the diagnostic cytomorphologic stain comprises Diff-Quik stain, Papanicolaou stain, Wright-Giemsa stain, hematoxylin/eosin stain, or a derivative or modification thereof.

35. The population of urinary exfoliated cells of any one of alternatives 25-34, wherein the diagnostic cytomorphologic stain is free of or has a reduced concentration of alcohol, such as methanol and/or ethanol

36. The population of urinary exfoliated cells of any one of alternatives 25-35, wherein the population of urinary exfoliated cells are fixed and/or permeabilized.

37. The population of urinary exfoliated cells of alternative 36, wherein the population of urinary exfoliated cells are fixed in methanol, ethanol, formaldehyde, or paraformaldehyde.

38. The population of urinary exfoliated cells of any one of alternatives 25-37, wherein the population of urinary exfoliated cells comprise bladder cancer cells.

39. The population of urinary exfoliated cells of alternative 38, wherein the bladder cancer cells comprise one or more bladder cancer specific biomarkers selected from the group consisting of S100P, p63, M344, LDQ10, 19A211, GATA-3, Ki-67, p16, Her-2, PD-L1, CTLA4, CK-17, CK-20, nmp-22, BTA, hTERT, and MCM5.

40. The population of urinary exfoliated cells of alternative 38 or 39, wherein the bladder cancer cells are urothelial carcinoma, squamous cell carcinoma, adenocarcinoma, small cell carcinoma, or sarcoma cells.

41. The population of urinary exfoliated cells of any one of alternatives 25-40, wherein the population of urinary exfoliated cells are mammalian.

42. The population of urinary exfoliated cells of any one of alternatives 25-41, wherein the population of urinary exfoliated cells are human.

43. The population of urinary exfoliated cells of any one of alternatives 25-42, wherein the immunocytochemistry or chromogenic in-situ hybridization stain, or both, and/or the diagnostic cytomorphologic stain comprises centrifugation or use of one or more inserts or transwells, wherein the one or more inserts or transwells comprises a porous membrane that permits aqueous solution to pass but retains the urinary exfoliated cells and, optionally wherein the one or more inserts or transwells further comprise a flange, protrusion, tab, or handle.

43. Urinary exfoliated cells stained by the method of any one of alternatives 1-23.

Other exemplary alternatives are described by the following additional embodiments:

44. A method for improved immunohistochemical staining and/or nucleic acid chromogenic in situ hybridization staining on a cell sample, comprising:
a) preparing a cell suspension of the cell sample in a container;
b) performing immunohistochemical staining and/or nucleic acid chromogenic in situ hybridization staining on the cells in the cell suspension in the container, comprising:
   i) adding a primary antibody and/or a primary nucleic acid probe that specifically binds to an antigen and/or a nucleic acid marker on the cells to the cell suspension to induce specific binding of the primary antibody and/or the primary nucleic acid probe to the antigen and/or the nucleic acid marker;
   ii) removing the unbound primary antibody and/or primary nucleic acid probe, and then preparing the cell suspension again,
   iii) adding a secondary antibody conjugated with a chromogenic enzyme and/or a secondary nucleic acid probe labeled with a chromogenic enzyme to the cell suspension to specifically bind to the primary antibody and/or the primary nucleic acid probe;
   iv) removing the unbound secondary antibody conjugated with the chromogenic enzyme and/or secondary nucleic acid probe labeled with the chromogenic enzyme, and then preparing the cell suspension again,
   v) adding a chromogenic substrate to the cell suspension for color development; during the color development process, constantly agitating the cells suspended in the cell suspension to uniformly disperse the chromogenic precipitates produced by catalysis to avoid concentrated accumulation;
   vi) removing excess chromogenic substrate and then preparing the cell suspension again; and
c) smearing the cell suspension on a slide.

45. The method of alternative 44, wherein in steps ii), iv) and vi), removing the unbound primary antibody and/or the primary nucleic acid probe, removing the unbound secondary antibody conjugated with the chromogenic enzyme and/or secondary nucleic acid probe labeled with the chromogenic enzyme and removing the excess chromogenic substrate are carried out by the following steps:
i) washing the cells;
ii) centrifuging to precipitate the cells; and
iii) discarding the supernatant.

46. The method of alternative 44,
wherein the container comprises one or more inserts or transwells and the cell suspension, preferably comprising urinary exfoliated cells, is placed in the one or more transwells of the container, wherein the one or more inserts or transwells comprises a porous membrane that permits aqueous solution to pass but retains the cells in the cell suspension, such as a porous membrane having a pore size of 1, 2, 3, 4, 5, 6, 7, or 8 µm or having a pore size within a range defined by any two of the aforementioned pore sizes and, optionally wherein the one or more inserts or transwells further comprise a flange, protrusion, tab, or handle;
and wherein in steps ii), iv) and vi), removing the unbound primary antibody and/or the primary nucleic acid probe, removing the unbound secondary antibody conjugated with the chromogenic enzyme and/or secondary nucleic acid probe labeled with the chromogenic enzyme and removing the excess chromogenic substrate are carried out by washing the cells in the one or more inserts or transwells with a wash buffer, wherein the wash buffer passes through the one or more inserts or transwells.

47. The method of any one of alternatives 44 to 46, wherein the cell sample is selected from a cytopathological fine needle aspiration (FNA) sample, a blood circulating tumor cell sample, a cervical scrape cell sample, and a urine exfoliated cell sample, or the cell sample is obtained by diluting a cell precipitate of a sample from a patient selected from body fluid, blood, serum, plasma, urine, saliva, sweat, sputum, semen, mucus, tear, lymph, amniotic fluid, pleural fluid, ascites, interstitial fluid, lung lavage fluid, cerebrospinal fluid, feces and a tissue sample.

48. The method of any one of alternatives 44 to 47, wherein the primary antibody and/or primary probe is a combination of multiple antibodies and/or probes, which respectively stain cell membrane, cytoplasm, and/or cell nucleus.

49. Suspension of stained ex vivo cells, wherein said cells have cell biomarker staining.

50. The suspension of stained ex vivo cells of alternative 49, wherein the cell is derived from a cell sample selected from the group consisting of a cytopathological fine needle aspiration (FNA) sample, a blood circulating tumor cell sample, a cervical scrape cell sample, a urine exfoliated cell sample, body fluid, blood, serum, plasma, urine, saliva, sweat, sputum, semen, mucus, tear, lymph, amniotic fluid, pleural fluid, ascites, interstitial fluid, lung lavage fluid, cerebrospinal fluid, feces and a tissue sample.

51. The suspension of stained ex vivo cells of alternative 50, wherein the cells are tumor cells, which have tumor marker staining.

52. The suspension of stained ex vivo cells of any one of alternatives 49 to 51, which is obtained by the method of any one of alternatives 44 to 48.

53. A pathological slide comprising the suspension of cells of any one of alternatives 49 to 52.

54. A multiple staining method for preparing immunohistochemical staining and/or nucleic acid chromogenic in situ hybridization staining in combination with cell morphological staining that can be used for pathological diagnosis on a cell sample, comprising:
a) preparing a cell suspension of the cell sample in a container;
b) performing immunohistochemical staining and/or nucleic acid chromogenic in situ hybridization staining on the cells in the cell suspension in the container, comprising:
   i) adding a primary antibody and/or a primary nucleic acid probe that specifically binds to an antigen and/or a nucleic acid marker on the cells to the cell suspension to induce specific binding of the primary antibody and/or the primary nucleic acid probe to the antigen and/or the nucleic acid marker;
   ii) removing the unbound primary antibody and/or primary nucleic acid probe, and then preparing the cell suspension again,
   iii) adding a secondary antibody conjugated with a chromogenic enzyme and/or a secondary nucleic acid probe labeled with a chromogenic enzyme to the cell suspension to specifically bind to the primary antibody and/or the primary nucleic acid probe;
   iv) removing the unbound secondary antibody conjugated with the chromogenic enzyme and/or secondary nucleic acid probe labeled with the chromogenic enzyme, and then preparing the cell suspension again,
   v) adding a chromogenic substrate to the cell suspension for color development; and during the color development process, constantly agitating the cells suspended in the cell suspension to uniformly disperse the chromogenic precipitates produced by catalysis to avoid concentrated accumulation;
   vi) removing excess chromogenic substrate and then preparing the cell suspension again; and
c) performing cell morphological staining that can be used for pathological diagnosis on the cells in the container, and then smearing the multiply stained cells on a pathological slide; or, smearing the immunohistochemically stained and/or nucleic acid chromogenic in situ hybridization stained cells on a pathological slide to perform cell morphological staining that can be used for pathological diagnosis.

55. The method of alternative 54, wherein in steps ii), iv) and vi), removing the unbound primary antibody and/or the primary nucleic acid probe, removing the unbound secondary antibody conjugated with the chromogenic enzyme and/or secondary nucleic acid probe labeled with the chromogenic enzyme, and removing the excess chromogenic substrate are carried out by the following steps:
i) washing the cells;
ii) centrifuging to precipitate the cells; and
iii) discarding the supernatant.

56. The method of alternative 54,
wherein the container comprises one or more inserts or transwells and the cell suspension, preferably comprising urinary exfoliated cells, is placed in the one or more inserts or transwells of the container, wherein the one or more inserts or transwells comprise a porous membrane that permits aqueous solution to pass but retains the cells in the cell suspension, such as a porous membrane having a pore size of 1, 2, 3, 4, 5, 6, 7, or 8 µm or having a pore size within a range defined by any two of the aforementioned pore sizes and, optionally wherein the one or more inserts or transwells further comprise a flange, protrusion, tab, or handle;
and wherein in steps ii), iv) and vi), removing the unbound primary antibody and/or the primary nucleic acid probe, removing the unbound secondary antibody conjugated with the chromogenic enzyme and/or secondary nucleic acid probe labeled with the chromogenic enzyme and removing the excess chromogenic substrate are carried out by washing the cells in the one or more inserts or transwells with a wash buffer, wherein the wash buffer passes through the one or more inserts or transwells.

57. The method of any one of alternatives 54 to 56, wherein the cell morphological staining that can be used for pathological diagnosis is selected from Diff-Quik staining, Papanicolaou staining, Wright-Giemsa staining, and hematoxylin/eosin (H&E) staining.

58. The method of alternative 57, wherein the cell morphological staining that can be used for pathological diagnosis is Diff-Quik staining.

59. The method of any one of alternatives 54 to 58, wherein the cell sample is selected from a cytopathological fine needle aspiration (FNA) sample, a blood circulating tumor cell sample, a cervical scrape cell sample, and a urine exfoliated cell sample, or the cell sample is obtained by diluting a cell precipitate of a sample from a patient selected from body fluid, blood, serum, plasma, urine, saliva, sweat, sputum, semen, mucus, tear, lymph, amniotic fluid, interstitial fluid, pleural fluid, ascites, lung lavage fluid, cerebrospinal fluid, feces and a tissue sample.

60. The method of any one of alternatives 54 to 59, wherein the primary antibody and/or primary probe is a combination of multiple antibodies and/or probes, which respectively stain cell membrane, cytoplasm, and/or cell nucleus.

61. A multiply stained ex vivo cell, which has both morphological staining that can be used for pathological diagnosis and cell biomarker staining on the same targeted cells.

62. The ex vivo cell of alternative 61, wherein the cell is derived from a cell sample selected from the group consisting of a cytopathological FNA sample, a blood circulating tumor cell sample, a cervical scrape cell sample, a urine exfoliated cell sample, body fluid, blood, serum, plasma, urine, saliva, sweat, sputum, semen, mucus, tear, lymph, amniotic fluid, interstitial fluid, pleural fluid, ascites, lung lavage fluid, cerebrospinal fluid, feces and a tissue sample.

63. The ex vivo cell of alternative 61, wherein the cell is a tumor cell, which has both morphological staining that can be used for pathological diagnosis and tumor marker staining on the same targeted cells.

64. The ex vivo cell of any one of alternatives 61 to 63, wherein the cell is obtained by the method of any one of alternatives 54 to 60.

65. A pathological slide comprising the multiply stained ex vivo cell of any one of alternatives 61 to 64.

66. A multiple staining kit for performing immunohistochemical staining and/or nucleic acid chromogenic in situ hybridization staining and cell morphological staining that can be used for pathological diagnosis of a cell sample, which comprises a reagent for immunohistochemical staining and/or nucleic acid chromogenic in situ hybridization staining on cell samples, and a reagent for cell morphological staining.

67. The kit of alternative 66, wherein the reagent for immunohistochemical staining and/or nucleic acid chromogenic in situ hybridization staining of a cell sample comprises a primary antibody and/or a primary nucleic acid probe, a secondary antibody conjugated with a chromogenic enzyme and/or a secondary nucleic acid probe labeled with a chromogenic enzyme, and a chromogenic substrate.

68. The kit of alternative 66 or 67, wherein the reagent used for cell morphological staining is selected from a reagent used for Diff-Quik staining, Papanicolaou staining, Wright-Giemsa staining, and H&E staining.

69. The kit of alternative 67, wherein the primary antibody and/or primary probe is a combination of multiple antibodies and/or probes, which respectively stain cell membrane, cytoplasm, and/or cell nucleus.

70. The kit of any one of alternatives 66 to 69, wherein the cell sample is selected from a cytopathological FNA sample, a tumor cell sample, a cervical scrape cell sample, and a urine exfoliated cell sample, or the cell sample is obtained by diluting a cell precipitate of a sample from a patient selected from body fluid, blood, serum, plasma, urine, saliva, sweat, sputum, semen, mucus, tear, lymph, amniotic fluid, interstitial fluid, pleural fluid, ascites, lung lavage fluid, cerebrospinal fluid, feces and a tissue sample.

71. The kit of any one of alternatives 66-70 further comprising a container, such as a plate, multi-well plate, or microtiter plate, e.g., having 1, 2, 4, 6, 8, 12, 24, 48, 64, 96 or 384 wells, and one or more inserts or transwells configured to fit into the well or wells of said container, wherein the one or more inserts or transwells comprises a porous membrane or screen that permits aqueous solution to pass but retains the cells of the cell sample, such as a porous membrane or screen having a pore size of 1, 2, 3, 4, 5, 6, 7, or 8 µm or having a pore size within a range defined by any two of the aforementioned pore sizes and, optionally wherein the one or more inserts or transwells further comprise a flange, protrusion, tab, or handle.

72. An insert or transwell comprising a porous membrane or screen that permits aqueous solution to pass but retains cells, preferably comprising urinary exfoliated cells, such as a porous membrane or screen having a pore size of 1, 2, 3, 4, 5, 6, 7, or 8 µm or having a pore size within a range defined by any two of the aforementioned pore sizes, wherein said insert or transwell is configured to fit within a well of a plate, multi-well plate, or microtiter plate, such as a plate, multi-well plate, or microtiter plate having 1, 2, 4, 6, 8, 12, 24, 48, 64, 96 or 384 wells and, optionally wherein the insert or transwell further comprises a flange, protrusion, tab, or handle.

73. A plate, multi-well plate, or microtiter plate comprising one or more inserts or transwells configured to fit the well or wells of said plate, multi-well plate, or microtiter plate, wherein said one or more inserts or transwells comprise a porous membrane or screen that permits aqueous solution to pass but retains cells, preferably comprising urinary exfoliated cells, such as a porous membrane or screen having a pore size of 1, 2, 3, 4, 5, 6, 7, or 8 µm or having a pore size within a range defined by any two of the aforementioned pore sizes and, optionally wherein the one or more inserts or transwells further comprise a flange, protrusion, tab, or handle.

74. The kit of alternative 71, insert or transwell of alternative 72, or plate, multi-well plate, or microtiter plate of alternative 73, wherein a plurality of inserts or transwells are provided and said plurality of inserts or transwells are connected or joined to each other and, optionally wherein the plurality of inserts or transwells further comprise a flange, protrusion, tab, or handle.

### BRIEF DESCRIPTION OF THE DRAWINGS

In addition to the features described herein, additional features and variations will be readily apparent from the following descriptions of the drawings and exemplary embodiments. It is to be understood that these drawings depict embodiments and are not intended to be limiting in scope.

FIGS. 1A-G depicts exemplary photomicrographs showing urinary sediment cells stained with 10% Diff-Quik or 5% Papanicolaou stain and liquid-based immunocytochemistry simultaneously. All urinary sediment cells are stained in a satisfactory manner to enable cytopathologic diagnosis. Cytopathologically atypical cells (e.g. bladder cancer cells) are stained with chromogenic precipitation (marked by arrows). Each of FIGS. 1A-G involve urine samples from different patients and therefore the morphology of the atypical cells look different. Immunocytochemistry was performed with commercially available mouse or rabbit monoclonal antibodies specific for human cytokeratin 20 (CK-20) (FIG. 1A), bladder tumor antigen (BTA) (FIG. 1B), p63 (FIG. 1C), hTERT (FIG. 1D), S100P (FIG. 1E), PD-L1 (FIG. 1F), or MCM5 (FIG. 1G) as the primary antibody (see e.g., LBP Med Sci & Tech and Abcam).

FIG. 2 depicts a photomicrograph showing urinary sediment cells stained by Diff-Quik and chromogenic in-situ hybridization simultaneously. All urinary sediment cells are stained in a satisfactory manner to enable cytopathologic diagnosis. There are four bladder cancer cells (bounded in the black box) that stain with chromogenic precipitation using human GATA-3 as the primary nucleic acid probe.

FIG. 3 depicts exemplary antibodies against bladder cancer biomarkers that can be used for immunocytochemistry in any one of the methods disclosed herein.

FIGS. 4A-B depicts exemplary photomicrographs showing urinary sediment cells stained with 5% Papanicolaou stain and liquid-based immunocytochemistry simultaneously. All urinary sediment cells are stained in a satisfactory manner to enable cytopathologic diagnosis. Cytopathologically atypical cells (e.g. bladder cancer cells) are stained with chromogenic precipitation (marked by arrows). Each of FIGS. 4A-B involve urine samples from different patients and therefore the morphology of the atypical cells look different. Immunocytochemistry was performed with commercially available mouse or rabbit monoclonal antibodies specific for human cytokeratin-17 (CK-17) (4A) or Her-2 (4B) as the primary antibody.

FIGS. 5A-D depict embodiments of a transwell or insert device for use in the staining of urinary exfoliated cells. The images depicted in FIGS. 5A-D are non-limiting, and other suitable embodiments of a transwell or insert device is envisioned.

### DETAILED DESCRIPTION

Cytopathology studies the causes and pathogenesis of diseases, as well as changes in the physiological functions of cells during the occurrence of diseases mainly based on abnormal conditions within cells, so as to propose the basis for diagnosis, prevention, and treatment of diseases. Clinical samples cover exfoliative cytology, fine needle aspiration cytology, blood circulating tumor cells, and other cytology (e.g. cytology during surgery, bone marrow, peripheral blood cytology, and AIDS cytology).

Conventional staining methods for cytopathology include Papanicolaou stain (Pap stain), Wright-Giemsa stain, and Diff-Quik stain. Diff-Quik, which is presently the most utilized stain for daily cytopathologic practice and recommended by the World Health Organization (WHO), usually contains a xanthene dye (e.g. Eosin Y), and a thiazine dye (e.g. methylene blue or azure A). It highlights nuclear features such as the shape and size of nuclei and nucleoli; the density of chromatin; cytoplasmic elements such as mucins, fat droplets and neurosecretory granules; and cell membrane features such as membrane grooves, projections, and vacuoles. Extracellular substances, such as free mucin, colloids, and ground substance, are also easily stained. These features are essential for making cytopathologic diagnoses.

The present disclosure relates to the areas of bladder cancer primary diagnosis, recurrent detection, disease progression, and disease screening, such as among high risk populations. The methods disclosed herein are used to evaluate cytopathologic slides harboring urinary sediment cells, including exfoliated malignant cells in the case of bladder cancer.

The unique feature of the methods provided herein is that pathologists, for the first time, can evaluate both cytopathologically qualified morphologic characteristics and bladder cancer specific biomarker expression on the same individual urinary sediment cell on the same slide. The combined evaluation is achieved through conventional light microscopy by pathologists and enables primary diagnosis of bladder cancer, monitoring of bladder cancer resurgence, and screening of bladder cancer using urine samples.

Urine cytology is a process of evaluating urinary sediment cells (and constituent malignant cells) on glass slides by pathologists, who examine the cytopathologic features of every individual cell under conventional light microscopy. It is routinely used in clinical practice as a noninvasive test for bladder cancer. It is highly effective for the detection of high-grade and high-stage disease with a reported high sensitivity and specificity for high-grade bladder cancer. However, it proves relatively ineffective as a tool to detect low-grade malignancy with reported sensitivity ranging only around 4% to 31% for the detection of low-grade tumors.

Cytology is commonly used to monitor high-grade and high-stage bladder cancer recurrence. However, urine cytology is not approved to be effective for low-grade bladder cancer, nor for bladder cancer screening among high risk populations because it lacks satisfactory sensitivity and/or specificity for bladder cancer detection.

In conventional methods, urinary sediment cells are obtained through 20-25 mL of urine sample by centrifugation. The sediment cells are then prepared as a thin layer of cells on pathology glass slides and stained with clinically approved methods to demonstrate the cellular morphologic features for pathologist to make diagnoses.

The bladder cancer grade describes how much cancer cells look like healthy cells when viewed under a microscope after staining. However, when pathologists see low grade bladder cancer cells, they often misinterpret them as normal cells, or run into a dilemma and categorize those cells as atypical or suspicious cells without definitive diagnosis. Because the apparent deficiencies and exceedingly high false negative rate for low-grade bladder tumors, other technologies have been explored to aid urine cytology. Bladder tumor specific biomarker expression on exfoliated malignant cells has gained much attention for this purpose. Two main technique platforms have been developed to meet this growing need:

1) Quantification of bladder tumor specific biomarker protein concentrations in urine samples.

2) Detection of biomarker expression in urine sediment cells by fluorescent techniques, namely immunofluorescent staining (IF) and fluorescent in-situ hybridization (FISH).

The US FDA so far has approved 6 products to detect bladder tumors using urinary samples, as depicted in Table 1. However, they have not been considered as better choices than urine cytology because of suboptimal sensitivity and specificity, and are not widely adopted by urologists Values for Table 1 adapted from Tabayoyong W & Kamat AM. Current Use and Promise of Urinary Markers for Urothelial Cancer. Curr Urol Rep. 2018;19(12):96. Predicted values (*) for the methods described herein (“Cytobay”) are expected based on clinical trials.

TABLE 1

Present methods vs. FDA-approved urinary biomarkers for the detection of bladder cancer

| Test | Type of Assay | Sensitivity | Specificity |
|---|---|---|---|
| BTA stat | Single-step office based | 57-83% | 60-92% |
| BTATRAK | Sandwich immunoassay | 66% | 65% |
| NMP22 BC | Lab-based ELISA | 47-100% | 78% |
| NMP22 BladderChek | Point-of-care | 47-100% | 78% |
| ImmunoCyt/uCyt | Cytology plus immunofluorescence | 50-100% | 69-79% |
| UroVysion | Fluorescence in situ hybridization | 41-70% | 80% |
| CytoBay | Diagnostic cytology plus immunocytochemistry | >95%* | >95%* |

Among the FDA-approved test in Table 1, the first 4 (BTA stat, BTATRAK, NMP22 BC, NMP22 BladderChek) measure the protein concentration of bladder cancer specific biomarkers, while ImmunoCyt/uCyt and UroVysion detect the biomarkers by immunofluorescence. None of them can provide a detailed cellular morphology that is qualified for pathologic diagnosis. Although immunofluorescent stains can delineate the overall shape and size of targeted cells, they are not enough nor qualified to make morphologic diagnoses by pathologists. Accordingly, no detailed morphologic or cytopathologic information that is qualified for pathologic diagnosis can be generated from these assays.

Furthermore, as shown in Table 1, FDA-approved techniques exhibit significant inter-observer discrepancies in sensitivity and specificity, which are two important parameters to evaluate the precision of the tests. The reduction in specificity is due to a large number of false positive results from benign or alternative causes including infection, stones, hematuria, and recent instrumentation of the urinary system. Widespread use of these assays has also been limited due to the requirement of special laboratory equipment (such as darkrooms and expensive fluorescent microscopes) and experienced readers to interpret test results.

Although alternatives to cytopathological staining, such as immunocytochemical and/or chromogenic in-situ hybridization stains are much needed for more accurate diagnoses, it is exceedingly difficult to perform these techniques with urinary sediment cells. These techniques are not used as adjunct technologies in urine cytology for several reasons:

1) Currently, urine cytology slides are prepared from 20-25 mL of urine sample. The cellular quantity in the urine sample is small and appropriate for one cytomorphologically stained slide. Routine cytomorphologic stains such as Diff-Quik impede the binding of primary antibodies or nucleic acid probes to their epitopes or targeted sequence because of the dyes using in the routine cytology stain. These dyes must be stripped off from the urinary sediment cells before immunostaining procedures can be successfully performed.

2) Even after stripping the dye, higher concentrations of primary antibodies or nucleic acid probes must be applied for a satisfactory staining. This may result in false positive results and misleading clinical diagnoses, disease monitoring, or treatment regimen selection.

3) The urinary sediment cells on the glass slides are exposed to an open environment during immunostaining procedures, which have multiple steps of changing reagents and washing. Some sediment cells are inevitably detached from the slides when changing reagents and washing solutions, and consequently lost with the discarded solutions. This may result in lower sensitivity and inaccurate results.

4) In the rare situation when stripping routine morphology stains is not necessary, the cytomorphology stain is inevitably deteriorated during the process of immunocytochemistry and/or chromogenic in-situ hybridization, hindering an accurate morphologic evaluation.

5) Chromogen precipitation on the stationary glass slides as the signals for immunocytochemistry or chromogenic in-situ hybridization may accumulate on the targeted cells to a point where cellular morphologic features are masked. This may result in suboptimal cytopathologic evaluation by pathologists.

6) Urinary sediment cells are attached to glass slides. The staining and washing reagents cannot penetrate to where the cells are attached to the slides (otherwise the cells would detach and be lost). This creates staining “blind spots” where the urinary sediment cells are attached to the glass slides. In addition, there are many narrow grooves between each attached cell and the glass slide surface. As a result, staining components are easily lodged into or stick in those hard-to-reach narrow grooves, creating the infamous “edge effects” around each sediment cell during immunocytochemistry or chromogenic in-situ hybridization. The side effects may result in false positive signals, hindering an accurate detection.

Therefore, current technology only provides for one-dimensional information on exfoliated malignant cells in urine, either cytomorphology (urine cytology) or bladder cancer specific biomarkers. Bladder cancer specific biomarker detection, whether by protein concentration or immunofluorescence, yields none or little morphologic information. To improve the sensitivity and specificity of urinary sediment cell diagnosis for bladder cancer, innovative methods, which combine diagnostically qualified cytomorphology with bladder cancer specific biomarker expression on the same individual cells are disclosed herein. The synergy obtained by the detection of cytomorphology and tumor markers on the same individual cells on the same slide facilitates and expedites analysis by pathologists, which generates more accurate cytopathologic diagnosis.

### Definitions

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, technical and scientific terms used herein have the same meaning as commonly understood when read in light of the instant disclosure by one of ordinary skill in the art to which the present disclosure belongs. For purposes of the present disclosure, the following terms are explained below.

The disclosure herein generally uses affirmative language to describe the numerous embodiments. The disclosure also includes embodiments in which subject matter is excluded, in full or in part, such as substances or materials, method steps and conditions, protocols, or procedures.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that varies by as much as 10% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “individual”, “subject”, or “patient” as used herein have their plain and ordinary meaning as understood in light of the specification, and mean a human or a non-human mammal, e.g., a dog, a cat, a mouse, a rat, a cow, a sheep, a pig, a goat, a non-human primate, or a bird, e.g., a chicken, as well as any other vertebrate or invertebrate. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, or guinea pigs, or the like.

The terms “effective amount” or “effective dose” as used herein have their plain and ordinary meaning as understood in light of the specification, and refer to that amount of a recited composition or compound that results in an observable effect. Actual dosage levels of active ingredients in an active composition of the presently disclosed subject matter can be varied so as to administer an amount of the active composition or compound that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

The terms “function” and “functional” as used herein have their plain and ordinary meaning as understood in light of the specification, and refer to a biological, enzymatic, or therapeutic function.

The term “inhibit” as used herein has its plain and ordinary meaning as understood in light of the specification, and may refer to the reduction or prevention of a biological activity. The reduction can be by a percentage that is, is about, is at least, is at least about, is not more than, or is not more than about, 10%, 20%, 30%, 40%, 50%, 60%, 70%, 80%, 90%, or 100%, or an amount that is within a range defined by any two of the aforementioned values. As used herein, the term “delay” has its plain and ordinary meaning as understood in light of the specification, and refers to a slowing, postponement, or deferment of a biological event, to a time which is later than would otherwise be expected. The delay can be a delay of a percentage that is, is about, is at least, is at least about, is not more than, or is not more than about, 0%, 10%, 20%, 30%, 40%, 50%, 60%, 70%, 80%, 90%, 100%, or an amount within a range defined by any two of the aforementioned values. The terms inhibit and delay may not necessarily indicate a 100% inhibition or delay. A partial inhibition or delay may be realized.

As used herein, the term “isolated” has its plain and ordinary meaning as understood in light of the specification, and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

As used herein, “in vivo” is given its plain and ordinary meaning as understood in light of the specification and refers to the performance of a method inside living organisms, usually animals, mammals, including humans, and plants, as opposed to a tissue extract or dead organism.

As used herein, “ex vivo” is given its plain and ordinary meaning as understood in light of the specification and refers to the performance of a method outside a living organism with little alteration of natural conditions.

As used herein, “in vitro” is given its plain and ordinary meaning as understood in light of the specification and refers to the performance of a method outside of biological conditions, e.g., in a petri dish or test tube.

The terms “nucleic acid” or “nucleic acid molecule” as used herein have their plain and ordinary meaning as understood in light of the specification, and refer to polynucleotides, such as deoxyribonucleic acid (DNA) or ribonucleic acid (RNA), oligonucleotides, those that appear in a cell naturally, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded. “Oligonucleotide” can be used interchangeable with nucleic acid and can refer to either double stranded or single stranded DNA or RNA. A nucleic acid or nucleic acids can be contained in a nucleic acid vector or nucleic acid construct (e.g. plasmid, virus, retrovirus, lentivirus, bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain elements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thereof.

A nucleic acid or nucleic acid molecule can comprise one or more sequences encoding different peptides, polypeptides, or proteins. These one or more sequences can be joined in the same nucleic acid or nucleic acid molecule adjacently, or with extra nucleic acids in between, e.g. linkers, repeats or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a nucleic acid as used herein has its plain and ordinary meaning as understood in light of the specification and refers to a sequence being after the 3′-end of a previous sequence, on the strand containing the encoding sequence (sense strand) if the nucleic acid is double stranded. The term “upstream” on a nucleic acid as used herein has its plain and ordinary meaning as understood in light of the specification and refers to a sequence being before the 5′-end of a subsequent sequence, on the strand containing the encoding sequence (sense strand) if the nucleic acid is double stranded. The term “grouped” on a nucleic acid as used herein has its plain and ordinary meaning as understood in light of the specification and refers to two or more sequences that occur in proximity either directly or with extra nucleic acids in between, e.g. linkers, repeats, or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths, but generally not with a sequence in between that encodes for a functioning or catalytic polypeptide, protein, or protein domain.

The nucleic acids described herein comprise nucleobases. Primary, canonical, natural, or unmodified bases are adenine, cytosine, guanine, thymine, and uracil. Other nucleobases include but are not limited to purines, pyrimidines, modified nucleobases, 5-methylcytosine, pseudouridine, dihydrouridine, inosine, 7-methylguanosine, hypoxanthine, xanthine, 5,6-dihydrouracil, 5-hydroxymethylcytosine, 5-bromouracil, isoguanine, isocytosine, aminoallyl bases, dye-labeled bases, fluorescent bases, or biotin-labeled bases.

The terms “peptide”, “polypeptide”, and “protein” as used herein have their plain and ordinary meaning as understood in light of the specification and refer to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein has its plain and ordinary meaning as understood in light of the specification and refers to a sequence being after the C-terminus of a previous sequence. The term “upstream” on a polypeptide as used herein has its plain and ordinary meaning as understood in light of the specification and refers to a sequence being before the N-terminus of a subsequent sequence.

The term “% w/w” or “% wt/wt” as used herein has its plain and ordinary meaning as understood in light of the specification and refers to a percentage expressed in terms of the weight of the ingredient or agent over the total weight of the composition multiplied by 100. The term “% v/v” or “% vol/vol” as used herein has its plain and ordinary meaning as understood in the light of the specification and refers to a percentage expressed in terms of the liquid volume of the compound, substance, ingredient, or agent over the total liquid volume of the composition multiplied by 100.

Bladder cancer can be categorized as either low-grade or high-grade. Low-grade tumors are characterized by cancerous cells that proliferate slowly and cytomorphologically resemble normal urothelial and other bladder cells. While low-grade bladder tumors do not often progress to more malignant and invasive tumors than high-grade bladder cancer, early detection and maintenance of low-grade tumors is important, as the presence of low-grade tumors may indicate an individual’s proclivity in developing advanced bladder cancer. High-grade tumors are ones that proliferate rapidly, may invade into the surrounding bladder muscle, and can eventually metastasize. While there are approved pathological methods to detect high-grade bladder tumors, the detection of low-grade tumors is difficult due to their similarity to normal bladder cells. A combination of cytomorphological and cancer biomarker assessment, as embodied in the methods of this disclosure, provides greater confidence in the identification of both low-grade and high-grade bladder cancer in patient samples.

The term “urinary exfoliated cells” as used herein refer to the small number of urothelial cells and other cells associated with the urinary tract, including potentially cancerous cells, are excreted during urine evacuation. Analyzing these excreted cells (e.g. cancerous cells, red blood cells, white blood cells, or bacteria) from urine samples provides a non-invasive method of assessing the health of an individual. However, the concentration of these urinary exfoliated cells is very low, and therefore enrichment is generally necessary. Typically, this is accomplished by centrifugation, such that the constituent cells are compacted into a cell pellet (termed “urinary sediment cells”), which can be resuspended and used for downstream processes.

Specific cellular targets in a sample can be detected and stained. These specific cellular targets may be any biological component, including but not limited to proteins or nucleic acids. Two approaches are immunohistochemistry (immunocytochemistry), which use antibodies to detect proteins (or any other epitope able to be bound by antibodies), and in-situ hybridization (fluorescent, chromogenic, or otherwise), which use nucleic acid probes to hybridize and detect nucleic acids such as DNA or RNA. Immunocytochemistry and chromogenic in-situ hybridization specific for bladder cancer specific biomarkers have been disclosed herein in some embodiments. In particular, chromogenic techniques which enzymatically convert chromogens to colored precipitates are used due to the ease of detection using conventional light microscopy. However, it is envisioned that alternative biomarker-specific stains and other biomarkers can be substituted by one skilled in the art.

Conventional immunocytochemistry is performed on glass slides. The cellular or tissue sample on the slides is incubated with a specific primary antibody for a protein target. After extensive washing to remove the primary antibody, the secondary antibody with conjugated chromogenic enzyme is incubated on the slides. Then, the corresponding chromogen is added to the slides, and positive signals are generated by chromogenic precipitation through catalytic reactions by the conjugated chromogenic enzyme. The signals on the slides can be evaluated by light microscopy.

Conventional chromogenic in-situ hybridization is performed on glass slides. The cellular or tissue sample on the slides is incubated with a specific nucleic acid probe that hybridizes with a nucleic acid target. After extensive washing to remove the nucleic acid probe, secondary reagents conjugated with chromogenic enzyme are sequentially incubated on the slides. Then, the corresponding chromogen is added to the slides, and positive signals are generated by chromogenic precipitation through catalytic reactions by the conjugated chromogenic enzyme. The signals on the slides can be evaluated by light microscopy.

The main differences of immunocytochemistry and chromogenic in-situ hybridization are the primary reagents (i.e. protein antibodies or nucleic acid probes). The signal production and evaluation are essentially the same.

The term “diagnostic cytomorphologic stain” as used herein refers to the staining process using a dye or a mixture of dyes which color cells and other biological material in a general manner (contrasting with immunocytochemistry or chromogenic in-situ hybridization stains which target a specific component). Diagnostic cytomorphologic stains are commonly used to improve contrast of cells under conventional light microscopy and to provide distinguishable detail regarding cell morphology for cytopathological determination. Examples of diagnostic cytomorphologic stains include Diff-Quik, Papanicolaou stain, Wright-Giemsa stain, and hematoxylin/eosin stain. It is envisioned that derivatives and modifications of known diagnostic cytomorphologic stains may be used for certain desirable properties, provided that the chemical dyes, which provide the color are maintained or substituted.

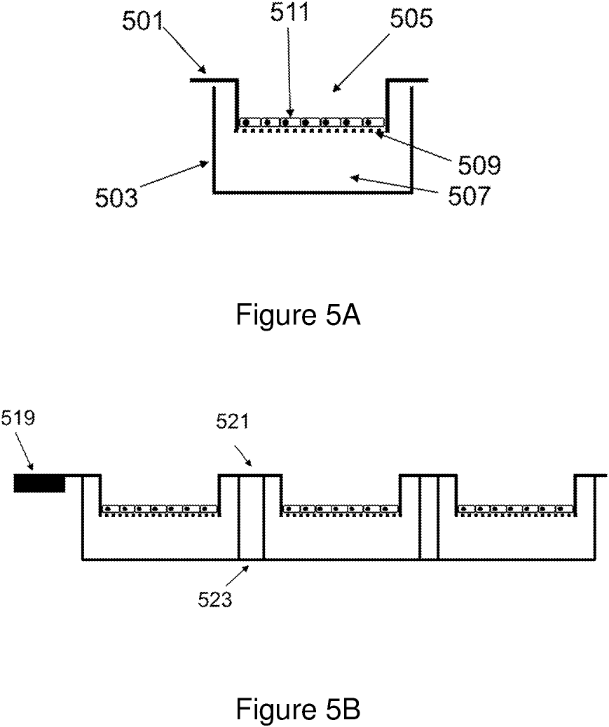

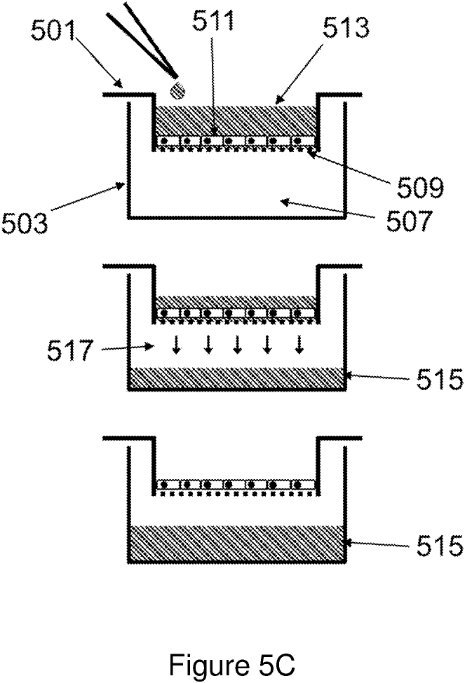

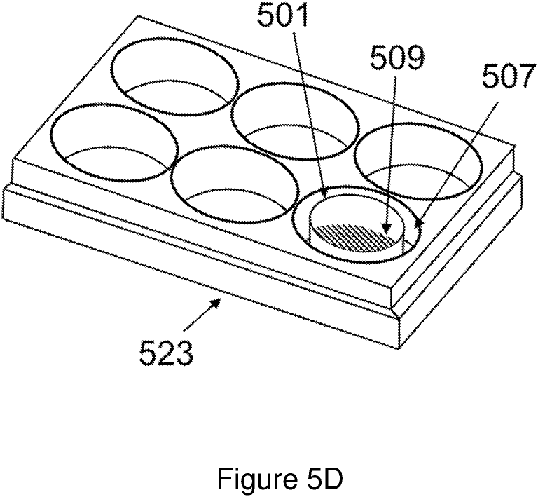

The term “transwell” or “insert” as used herein is understood in the art and refers to a device that has a porous membrane or screen and separates two regions based on the permeability of the porous membrane or screen. An embodiment of a transwell or insert device as used herein is depicted in FIGS. 5A-D. An embodiment of the transwell or insert is a well-shaped device 501 that comprises a porous membrane or screen 509 and can suitably fit a container 503 such that the container 503 has an lower compartment 507 where the transwell 501 is situated and the transwell 501 has an upper compartment 505, and where the lower compartment 507 and upper compartment 505 is interfaced across the porous membrane or screen 509, which may permit some level of transfer across based on particle size. In the context of use in cell culture, a population of cells 511, which may be in the form of a monolayer, that is situated on the porous membrane or screen 509 of the transwell 501 can be contacted with a growth medium in both the lower compartment and upper compartment. In some cases, molecules produced by the population of cells are able to permeate across the porous membrane or screen into the lower compartment, but the cells will remain in the upper compartment. These transwells may also be used as a method to contact a population of cells with a desired liquid composition for a brief period of time. For example, a population of cells 511 is situated on the porous membrane or screen 509 of the transwell 501, and a solution 513 (e.g. a wash buffer as part of a staining process) is applied to the population of cells 511, where the solution 513 contacts the cells 511 and then passes through the porous membrane or screen 509 in the process shown as 517 into the lower compartment as spent solution 515 (e.g. when using a wash buffer, to separate the population of cells from substances to be removed, such as dyes, proteins, antibodies, or nucleic acids). Typically, the transwell 501 will be outfitted with a porous membrane or screen 509 that has a pore size that is smaller than target cells such that the target cells are retained on the porous membrane or screen while particles smaller than the pore size are able to cross the membrane or screen (e.g. smaller cells, macromolecules, small molecules, solvent). In some embodiments, the porous membrane or screen will have a pore size of 1, 2, 3, 4, 5, 6, 7, or 8 µm in diameter. In some embodiments, the transwells are used to filter urinary exfoliated cells, which will be retained by a porous membrane or screen with a pore size of 1, 2, 3, 4, 5, 6, 7, or 8 µm in diameter. The transwell 501 is sized to suitably fit in an appropriate container 503. The container can be a well of a plate, multi-well plate, or microtiter plate, for example, a plate, multi-well plate, or microtiter plate having 1, 2, 4, 6, 8, 12, 24, 48, 64, 96, or 384 wells (e.g. such as a 6 well plate as embodied as 523). The transwell, and optionally the container, can be produced from any suitable material, such as a plastic (e.g. polycarbonate, polystyrene, PTFE, PET). The porous membrane or screen can be a different material than the rest of the transwell device according to the desired needs. In some embodiments, the transwell may also have a flange, protrusion, tab, or handle 519 to improve ease in moving the transwell 501 to and from the container 503. In some embodiments, the more than one transwells may be connected as one unit 521, and a suitable container 523 that contains the unit 521 can be used.

### Multiple Cytopathological Staining

Disclosed herein are methods of detecting or diagnosing bladder cancer, such as low-grade bladder cancer, from urine sediment cells isolated from urine samples. The methods comprise staining steps provided herein.

The methods disclosed herein overcome the limitations of conventional cytopathological detection processes and produce cytopathology slides harboring diagnostic cytomorphology and tumor marker expression on the same urinary sediment cells. By enabling multiple stains on the same cells, a pathologist is able to more confidently assess cytological properties suggesting potentially abnormal or cancerous cells. Sediment cells are harvested in a container (e.g. test tube), and then the cells are stained in a liquid suspension. The urinary sediment cells are smeared or prepared as a thin layer of cells and fixed onto glass slides after one or more desired staining procedures are finished. This approach makes multiple stains (i.e. morphologic and tumor biomarker stains) feasible. When a change of reagent is required during staining, the quantity of urine sediment cells are preserved, for example, by centrifuging the cells and discarding the cell-free supernatants containing staining reagents or using one or more inserts or transwells, which can be connected or joined, so that the plurality of inserts or transwells can be inserted or removed from a plurality of wells simultaneously. Optionally, a flange, protrusion, tab, or handle is provided on the insert or transwell or plurality of inserts or transwells, e.g., connected to a rim or lip of the one or more inserts or transwells or the stem, which connects a plurality of inserts or transwells, to facilitate moving the one or more inserts or transwells from the wells. In some embodiments, the one or more inserts or transwells comprise a porous membrane or screen that permits aqueous solution to pass but retains cells, preferably comprising urinary exfoliated cells, such as a porous membrane or screen having a pore size of 1, 2, 3, 4, 5, 6, 7, or 8 µm or having a pore size within a range defined by any two of the aforementioned pore sizes, wherein said insert or transwell is configured to fit within a well of a plate, multi-well plate, or microtiter plate, such as a plate, multi-well plate, or microtiter plate having 1, 2, 4, 6, 8, 12, 24, 48, 64, 96 or 384 wells. In some embodiments, the use of one or more inserts or transwells retains cell morphology better than centrifugation. By performing the staining in suspension, incomplete staining, which may lead to blind spots or edge effects, is avoided. Furthermore, as staining is not performed on glass slides, the risk of nonspecific chromogen precipitation as background noise is avoided. Precipitated chromogen signals are uniformly distributed within the target cells and does not interfere with morphologic evaluation of cellular details. In addition, by performing morphologic staining with modified solutions containing reduced or no methanol and/or ethanol (or other alcohol or fixative), interference on chromogenic staining is mitigated.

Briefly, the procedures for cytopathological staining comprise fixing and/or permeabilizing urinary sediment cells in an aqueous cell suspension, performing immunocytochemistry and/or chromogenic in-situ hybridization stains, and either a) performing detailed morphologic staining that is qualified for cytopathologic diagnosis, and smearing or preparing a thin layer of the multiple stained urinary sediment cells on glass slides for microscopic evaluation, or b) smearing or preparing a thin layer of the immunocytochemistry and/or chromogenic in-situ hybridization stained urinary sediment cells on glass slides, and then performing detailed morphologic staining that is qualified for cytopathologic diagnosis. In some embodiments, the morphologic staining is performed using Diff-Quik, Papanicolaou stain, Wright-Giemsa stain, or hematoxylin/eosin stain, or derivatives or modifications thereof, such as modified stains comprising the principal staining components of the exemplary stains thereof.

Disclosed herein are methods of detecting bladder cancer in a patient. In some embodiments, the methods comprise isolating bladder cells from the patient, fixing the bladder cells in an aqueous cell suspension, staining the bladder cells with a immunocytochemical and/or chromogenic in-situ hybridization stain, staining the bladder cells with a diagnostic cytomorphologic stain, and assessing the bladder cells as comprising bladder cancer cells by the immunocytochemical and/or chromogenic in-situ hybridization and the diagnostic cytomorphologic stain, thereby detecting bladder cancer in the patient. In some embodiments, the bladder cells are urinary exfoliated cells. The cytology slides using the methodology described herein enable pathologists to make bladder cancer diagnosis by evaluating nuclear features such as the shape and size of nuclei and nucleoli; the density of chromatin; cytoplasmic elements such as mucins, fat droplets and neurosecretory granules; and cell membrane features such as membrane grooves, projections, and vacuoles. Extracellular substances, such as free mucin, colloids, and ground substance, are also easily evaluated. When a pathologist encounters difficulties in cytomorphology, they can further take advantage of bladder cancer specific tumor markers stained on the same slides using the methodology described herein. This is important because pathologists currently would miss 1 bladder cancer diagnosis from every 2-3 patients if based on the current urine cytology status alone. It is common knowledge that urine cytology sensitivity only ranges from 48%-70% for bladder cancer. It is worth noting that any false-positive tumor marker staining on non-cancer cells can be effectively excluded by combining cytomorphologic evaluation. Another reason the methodology described herein is superior is because the present application is based on chromogenic immunocytochemistry or in situ hybridization, which is generally believed to be more sensitive and accurate than fluorescence-based methods currently used in comparable assays such as ImmunoCyt/uCyt and UroVysion. Therefore, the methodology described herein can greatly increase the sensitivity and accuracy of the urine cytology, make urine samples become reliable, cost-effective, non-invasive and convenient sources for bladder cancer primary diagnosis, real-time disease surveillance and bladder cancer screening. Optionally, once the bladder cancer is identified in the patient sample, a clinical management strategy specific for the extent, type, or treatment of the bladder cancer is administered to said patient. For example, depending on the results of the analysis, it is contemplated that surgery and/or radiation and/or chemotherapy and/or immunotherapy and/or target therapy can be administered to said patient. For instance, should Ki-67 and p16 be detected using the methodology described herein, which indicates that the bladder cancer may progress more rapidly, more aggressive surgery and/or chemotherapy is administrated, and closer clinical follow-up should be warranted; should PD-L1 and CTLA4 be detected using the methodology described herein, one or more immunotherapeutic monoclonal agents is administered with or without intravesical chemotherapy; should Her-2 expression in bladder cancer cells be detected using the methodology described herein, targeted therapy such as Herceptin, Afatinib, RC48-ADC, DS8201a, or PRS-343 may be administrated. More importantly, by combining biomarker detection with diagnostic cytomorphology, any false positive tumor marker staining on non-cancer cells can be excluded, preventing unnecessary treatment or wasting of medical resources. Finally, with high sensitivity and specificity of the methodology described herein, bladder cancer can become the fifth type of cancer that can be effectively screened in high-risk population, after colorectal cancer, breast cancer, lung cancer, and cervical cancer.

In some embodiments, the bladder cancer cells used for the methodology described herein are isolated from a urine sample from the patient. In some embodiments, the urine sample comprises urinary exfoliated cells. In some embodiments, the bladder cells are urinary exfoliated cells isolated from the urine sample. In some embodiments, the urine sample comprises the urinary exfoliated cells at a concentration of urinary exfoliated cells of about 10, 100, 200, 300, 400, 500, 600, 700, 800, 900, 1000, 1100, 1200, 1300, 1400, 1500, 2000, 3000, 4000, 5000, 6000, 7000, 8000, 9000, or 10000 cells/mL of urine sample, or any concentration of urinary exfoliated cells within a range defined by any two of the aforementioned concentrations. The amount of cells in a urine sample is relatively low, which necessitates careful handling of the sample and the isolated cells thereof to minimize loss. In some embodiments, the urine sample is centrifuged or allowed to settle by gravity to gather the urinary exfoliated cells as urinary sediment cells. In some embodiments, the urinary exfoliated cells are urinary sediment cells isolated from the urine sample by centrifugation.

In some embodiments, the immunocytochemistry or chromogenic in-situ hybridization stain, or both is performed in an aqueous cell suspension. In some embodiments, the immunocytochemistry or chromogenic in-situ hybridization stain, or both is not performed on a solid substrate. In some embodiments, the immunocytochemistry or chromogenic in-situ hybridization stain, or both comprises labeling one or more bladder cancer specific biomarkers. In some embodiments, the one or more bladder cancer specific biomarkers are labeled as a protein or a nucleic acid (e.g. DNA or RNA). In some embodiments, the one or more bladder cancer specific biomarkers are selected from the group consisting of S100 calcium binding protein P (S100P), tumor protein p63 (p63), M344 tumor-associated antigen (detected by the antibody M344), mucin 2 (detected by the antibody LDQ10), carcinoembryonic antigen (CEA; detected by the antibody 19A211), GATA-binding protein 3 (GATA-3), Marker of Proliferation Ki-67 (Ki-67), p16, human epidermal growth factor receptor 2 (Her-2), Programmed death-ligand 1 (PD-L1), cytotoxic T-lymphocyte-associated protein 4 (CTLA4), cytokeratin-17 (CK-17), cytokeratin-20 (CK-20), nuclear matrix protein number 22 (nmp-22), bladder tumor antigen (BTA), telomerase reverse transcriptase (hTERT), and mini-chromosome maintenance protein 5 (MCM5).

In some embodiments, the immunocytochemistry or chromogenic in-situ hybridization stain, or both, comprise adding specific primary antibody and/or nucleic acid probe, incubating the primary antibody and/or nucleic acid probe to allow them to bind to their specific targets, removing unbound primary antibody and/or nucleic acid probe, adding secondary antibody and/or nucleic acid probe that is conjugated with a chromogenic enzyme, incubating the secondary antibody and/or nucleic acid probe to allow them to bind to the primary antibody and/or nucleic acid probe, removing unbound secondary antibody and/or nucleic acid probe, adding the chromogen to the cell suspension for the chromogenic reaction, mildly vibrating or agitating the cell suspension to ensure uniform chromogenic precipitation, and removing unused chromogen to stop the chromogenic reaction. In some embodiments, the unbound antibodies, nucleic acid probes and/or chromogen are removed by washing the urinary exfoliated cells, centrifuging the suspension to precipitate the urinary exfoliated cells, and discarding the cell-free supernatant. In some embodiments, the cell suspension is placed in one or more inserts or transwells comprising a porous membrane or screen that permits aqueous solution to pass but retains cells, preferably comprising urinary exfoliated cells, such as a porous membrane or screen having a pore size of 1, 2, 3, 4, 5, 6, 7, or 8 µm or having a pore size within a range defined by any two of the aforementioned pore sizes, wherein said insert or transwell is configured to fit within a well of a plate, multi-well plate, or microtiter plate, such as a plate, multi-well plate, or microtiter plate having 1, 2, 4, 6, 8, 12, 24, 48, 64, 96 or 384 wells, and the unbound antibodies, nucleic acid probes and/or chromogen are removed by passing a wash buffer through the one or more inserts or transwells having the cells. Optionally, a flange, protrusion, or handle is provided on the transwell or plurality of inserts or transwells, e.g., connected to a rim or lip of the one or more inserts or transwells or a stem that connects a plurality of inserts or transwells, to facilitate moving one or more transwells from the wells. In some alternative embodiments, the urinary exfoliated cells are passed through a microfluidic channel with high protein or nucleic acid affinity to remove the unbound antibodies and/or nucleic acid probes. In some embodiments, the chromogenic enzyme is horseradish peroxidase (HRP) or alkaline phosphatase (AP). In some embodiments, the chromogen is 3,3-diaminobenzidine (DAB), aminoethyl carbazole (AEC), 3,3′5,5′-tetramethylbenzidine (TMB), Fast Red, Permanent Red, nitro blue tetrazolium chloride (NBT), 5-bromo-4-chloro-3-indolyl phosphate (BCIP), StayYellow, StayGreen, or derivatives thereof, or any combination thereof. In some embodiments, the immunocytochemistry or chromogenic in-situ hybridization, or both, is specific for one or more bladder cancer specific biomarkers. In some embodiments, the immunocytochemistry or chromogenic in-situ hybridization, or both, is specific for a protein or nucleic acid selected from the group consisting of S100P, p63, M344, LDQ10, 19A211, GATA-3, Ki-67, p16, Her-2, PD-L1, CTLA4, CK-17, CK-20, nmp-22, BTA, hTERT, and MCM5.

In some embodiments, the diagnostic cytomorphologic stain is performed either in an aqueous cell suspension (before mounting), or after mounting the urinary exfoliated cells onto a solid substrate. In some embodiments, performing the diagnostic cytomorphologic stain in the aqueous cell suspension enables uniform diagnostic cytomorphologic staining of the urinary exfoliated cells and prevents blind spots and edge effects. In some embodiments, assessing the urinary exfoliated cells by the diagnostic cytomorphologic stain comprises assessing characteristics of the urinary exfoliated cells comprising nuclear features, shape or size of the nuclei or nucleoli; the density of chromatin; cytoplasmic elements such as mucins, fat droplets or neurosecretory granules; or cell membrane features such as membrane grooves, projections, or vacuoles. In some embodiments, the diagnostic cytomorphologic stain comprises Diff-Quik stain, Papanicolaou stain, Wright-Giemsa stain, hematoxylin/eosin stain, or a derivative or modification thereof. In some embodiments, the diagnostic cytomorphologic stain is free of or has a reduced concentration of an alcohol, such as methanol and/or ethanol. In some embodiments, the diagnostic cytomorphologic stain has a reduced concentration of an alcohol, such as methanol and/or ethanol, relative to conventionally used stains. In some embodiments, the solid substrate is a microscope slide. In some embodiments, the microscope slide is glass or plastic.

In some embodiments, the stained urinary exfoliated cells are assessed by light microscopy, digital pathology slides, or artificial intelligence. In some embodiments, the stained urinary exfoliated cells are assessed as comprising bladder cancer cells. In some embodiments, the urinary exfoliated cells are assessed as comprising bladder cancer cells by the immunocytochemistry or chromogenic in-situ hybridization stain, or both, and/or the diagnostic cytomorphologic stain. In some embodiments, the bladder cancer cells comprise one or more bladder cancer specific biomarkers selected from the group consisting of S100P, p63, M344, LDQ10, 19A211, GATA-3, Ki-67, p16, Her-2, PD-L1, CTLA4, CK-17, CK-20, nmp-22, BTA, hTERT, and MCM5. In some embodiments, the bladder cancer cells comprise morphological qualities that are representative of cancer cells.

In some embodiments, bladder cancer is detected in the patient. In some embodiments, the bladder cancer is urothelial carcinoma, squamous cell carcinoma, adenocarcinoma, small cell carcinoma, sarcoma, or any combination thereof. In some embodiments, the bladder cancer is a low-grade bladder cancer or a high-grade bladder cancer. In some embodiments, the bladder cancer is a low-grade bladder cancer.

In some embodiments, any of the methods disclosed herein further comprise treating the patient for bladder cancer. In some embodiments, treating the patient for bladder cancer comprises surgery, chemotherapy, immunotherapy, targeted therapy, or radiation therapy, or any combination thereof. In some embodiments, the chemotherapy comprises administration of cisplatin, carboplatin, fluorouracil, mitomycin, gemcitabine, methotrexate, vinblastine, doxorubicin, erdafitinib, afatinib, docetaxel, or paclitaxel, or any combination thereof. In some embodiments, the immunotherapy comprises administration of Bacillus Calmette-Guerin (BCG), atezolizumab, avelumab, durvalumab, enfortumab, nivolumab, ipilimumab, trastuzumab, disitamab, PRS-343, or pembrolizumab, or any combination thereof. In some embodiments, the targeted therapy comprises administration of targeted Herceptin, Afatinib, RC48-ADC, DS8201a, or PRS-343, or any combination of thereof.

In any of the embodiments provided herein, the patient is a mammal. In some embodiments, the patient is a human. In some embodiments, the patient is selected to provide urinary exfoliated cells based on presentation of clinical symptoms of bladder cancer and/or being part of a population at high risk of developing bladder cancer.

### Multiply Stained Urinary Exfoliated Cells

Also disclosed herein are the urinary exfoliated cells that are multiply stained. In some embodiments, the urinary exfoliated cells are multiply stained according to any of the methods disclosed herein.

Disclosed herein are populations of urinary exfoliated cells stained with a liquid-based immunocytochemistry or chromogenic in-situ hybridization stain, or both, and a diagnostic cytomorphologic stain. In some embodiments, the population of urinary exfoliated cells is isolated from a patient. In some embodiments, the urinary exfoliated cells are urinary sediment cells isolated from a urine sample from the patient by centrifugation. In some embodiments, the immunocytochemistry or chromogenic in-situ hybridization stain, or both, are performed by any of the methods and involve any of the staining steps disclosed herein. In some embodiments, immunocytochemistry or chromogenic in-situ hybridization stain, or both, has been performed in an aqueous cell suspension. In some embodiments, the population of urinary exfoliated cells is stained with the immunocytochemistry or chromogenic in-situ hybridization stain with one or more bladder cancer specific biomarkers. In some embodiments, the one or more bladder cancer specific biomarkers are selected from the group consisting of S100P, p63, M344, LDQ10, 19A211, GATA-3, Ki-67, p16, Her-2, PD-L1, CTLA4, CK-17, CK-20, nmp-22, BTA, hTERT, and MCM5. In some embodiments, the population of urinary exfoliated cells are stained for one or more bladder cancer specific biomarkers. In some embodiments, the population of urinary exfoliated cells are stained for one or more of S100P, p63, M344, LDQ10, 19A211, GATA-3, Ki-67, p16, Her-2, PD-L1, CTLA4, CK-17, CK-20, nmp-22, BTA, hTERT, or MCM5. In some embodiments, the diagnostic cytomorphologic stain has been performed either a) in an aqueous cell suspension or b) after the population of urinary exfoliated cells have been mounted onto a solid substrate. In some embodiments, the solid substrate is a microscope slide. In some embodiments, the diagnostic cytomorphologic stain comprises Diff-Quik stain, Papanicolaou stain, Wright-Giemsa stain, hematoxylin/eosin stain, or a derivative or modification thereof. In some embodiments, the diagnostic cytomorphologic stain is free of or has a reduced concentration of an alcohol, such as methanol and/or ethanol. In some embodiments, the population of urinary exfoliated cells are fixed and/or permeabilized. In some embodiments, the population of urinary exfoliated cells are fixed in methanol, ethanol, formaldehyde, or paraformaldehyde. In some embodiments, the population of urinary exfoliated cells comprise bladder cancer cells. In some embodiments, the bladder cancer cells comprise one or more bladder cancer specific biomarkers selected from the group consisting of S100P, p63, M344, LDQ10, 19A211, GATA-3, Ki-67, p16, Her-2, PD-L1, CTLA4, CK-17, CK-20, nmp-22, BTA, hTERT, and MCM5. In some embodiments, the bladder cancer cells are urothelial carcinoma, squamous cell carcinoma, adenocarcinoma, small cell carcinoma, or sarcoma cells. In some embodiments, the population of urinary exfoliated cells are mammalian. In some embodiments, the population of urinary exfoliated cells are human.

In some embodiments, the population of urinary exfoliated cells are stained by a method of immunocytochemistry or chromogenic in-situ hybridization staining, or both, the method comprising adding specific primary antibody and/or nucleic acid probe, incubating the primary antibody and/or nucleic acid probe to allow them to bind to their specific targets, removing unbound primary antibody and/or nucleic acid probe, adding secondary antibody and/or nucleic acid probe that is conjugated with a chromogenic enzyme, incubating the secondary antibody and/or nucleic acid probe to allow them to bind to the primary antibody and/or nucleic acid probe, removing unbound secondary antibody and/or nucleic acid probe, adding the chromogen to the cell suspension for the chromogenic reaction, mildly vibrating or agitating the cell suspension to ensure uniform chromogenic precipitation, and removing unused chromogen to stop the chromogenic reaction. In some embodiments, the unbound antibodies, nucleic acid probes and/or chromogen are removed by washing the urinary exfoliated cells, centrifuging the suspension to precipitate the urinary exfoliated cells, and discarding the cell-free supernatant. In some embodiments, the cell suspension is placed in one or more inserts or transwells comprising a porous membrane or screen that permits aqueous solution to pass but retains cells, preferably comprising urinary exfoliated cells, such as a porous membrane or screen having a pore size of 1, 2, 3, 4, 5, 6, 7, or 8 µm or having a pore size within a range defined by any two of the aforementioned pore sizes, wherein said one or more inserts or transwells is configured to fit within a well of a plate, multi-well plate, or microtiter plate, such as a plate, multi-well plate, or microtiter plate having 1, 2, 4, 6, 8, 12, 24, 48, 64, 96 or 384 wells, and the unbound antibodies, nucleic acid probes and/or chromogen are removed by passing a wash buffer through the one or more inserts or transwells having the cells. Optionally, a flange, protrusion, or handle is provided on the insert or transwell or plurality of inserts or transwells, e.g., connected to a rim or lip of the one or more inserts or transwells or a stem that connects a plurality of inserts or transwells, to facilitate moving one or more inserts or transwells from the wells. In some alternative embodiments, the urinary exfoliated cells are passed through a microfluidic channel with high protein or nucleic acid affinity to remove the unbound antibodies and/or nucleic acid probes. In some embodiments, the chromogenic enzyme is horseradish peroxidase (HRP) or alkaline phosphatase (AP). In some embodiments, the chromogen is 3,3-diaminobenzidine (DAB), aminoethyl carbazole (AEC), 3,3′5,5′-tetramethylbenzidine (TMB), Fast Red, Permanent Red, nitro blue tetrazolium chloride (NBT), 5-bromo-4-chloro-3-indolyl phosphate (BCIP), StayYellow, StayGreen, or derivatives thereof, or any combination thereof. In some embodiments, the immunocytochemistry or chromogenic in-situ hybridization, or both, is specific for one or more bladder cancer specific biomarkers. In some embodiments, the immunocytochemistry or chromogenic in-situ hybridization, or both, is specific for a protein or nucleic acid selected from the group consisting of S100P, p63, M344, LDQ10, 19A211, GATA-3, Ki-67, p16, Her-2, PD-L1, CTLA4, CK-17, CK-20, nmp-22, BTA, hTERT, and MCM5.

### Reagents and Kits

Also envisioned herein are multiple staining kit embodiments. In some embodiments, the multiple staining kits are used for performing immunohistochemical staining and/or nucleic acid chromogenic in-situ hybridization staining and cell morphological staining that can be used for pathological diagnosis of a cell sample. In some embodiments, the kit comprises a reagent for immunohistochemical staining and/or nucleic acid chromogenic in-situ hybridization staining on cell samples, and a reagent for cell morphological staining. In some embodiments, the reagent for immunohistochemical staining and/or nucleic acid chromogenic in-situ hybridization staining of a cell sample comprises a primary antibody and/or a primary nucleic acid probe, a secondary antibody conjugated with a chromogenic enzyme and/or a secondary nucleic acid probe labeled with a chromogenic enzyme, and a chromogenic substrate. In some embodiments, the reagent used for cell morphological staining is selected from a reagent used for Diff-Quik staining, Papanicolaou staining, Wright-Giemsa staining, and H&E staining. In some embodiments, the primary antibody and/or primary probe is a combination of multiple antibodies and/or probes, which respectively stain cell membrane, cytoplasm, and/or cell nucleus. In some embodiments, the cell sample is selected from a cytopathological FNA sample, a tumor cell sample, a cervical scrape cell sample, and a urine exfoliated cell sample, or the cell sample is obtained by diluting a cell precipitate of a sample from a patient selected from body fluid, blood, serum, plasma, urine, saliva, sweat, sputum, semen, mucus, tear, lymph, amniotic fluid, interstitial fluid, pleural fluid, ascites, lung lavage fluid, cerebrospinal fluid, feces and a tissue sample. In some embodiments, the kit further comprises a container, such as a plate, multi-well plate, or microtiter plate, e.g., having 1, 2, 4, 6, 8, 12, 24, 48, 64, 96 or 384 wells, and one or more inserts or transwells configured to fit into the well or wells of said container, wherein the one or more inserts or transwells comprises a porous membrane or screen that permits aqueous solution to pass but retains the cells of the cell sample, such as a porous membrane or screen having a pore size of 1, 2, 3, 4, 5, 6, 7, or 8 µm or having a pore size within a range defined by any two of the aforementioned pore sizes and, optionally wherein the one or more inserts or transwells further comprise a flange, protrusion, tab, or handle.

Also disclosed herein are inserts or transwells. In some embodiments, the insert or transwell comprises a porous membrane or screen that permits aqueous solution to pass but retains cells, preferably comprising urinary exfoliated cells, such as a porous membrane or screen having a pore size of 1, 2, 3, 4, 5, 6, 7, or 8 µm or having a pore size within a range defined by any two of the aforementioned pore sizes, wherein said insert or transwell is configured to fit within a well of a plate, multi-well plate, or microtiter plate, such as a plate, multi-well plate, or microtiter plate having 1, 2, 4, 6, 8, 12, 24, 48, 64, 96 or 384 wells and, optionally wherein the insert or transwell further comprises a flange, protrusion, tab, or handle.

Also disclosed herein are plates, multi-well plates, or microtiter plates. In some embodiments, the plate, multi-well plate, or microtiter plate comprises one or more inserts or transwells configured to fit the well or wells of said plate, multi-well plate, or microtiter plate, wherein said one or more inserts or transwells comprise a porous membrane or screen that permits aqueous solution to pass but retains cells, preferably comprising urinary exfoliated cells, such as a porous membrane or screen having a pore size of 1, 2, 3, 4, 5, 6, 7, or 8 µm or having a pore size within a range defined by any two of the aforementioned pore sizes and, optionally wherein the one or more inserts or transwells further comprise a flange, protrusion, tab, or handle.

In some embodiments, any of the kits, inserts or transwells, or plates, multi-well plates, or microtiter plates may comprise a plurality of inserts or transwells, where the plurality of inserts or transwells are connected or joined to each other and, optionally wherein the plurality of inserts or transwells further comprise a flange, protrusion, tab, or handle.

### Selection of Bladder Cancer Patients

In some embodiments, the urine sample and/or the constituent urinary sediment cells are obtained from a patient. In some embodiments, the patient is selected to provide a urine sample. In some embodiments, the patient is a mammal. In some embodiments, the patient is a human. In some embodiments, the patient has bladder cancer, is at risk of having bladder cancer, has previously had bladder cancer, or does not have bladder cancer. In some embodiments, the patient presents clinical symptoms of bladder cancer, including but not limited to hematuria (blood in urine), having to urinate more often than usual, pain or burning during urination, feeling a need to urinate even if their bladder is not full, having trouble urinating or having a weak urine stream, or having to urinate many times throughout the night.

In some embodiments, the patient is among a high-risk population for bladder cancer. In some embodiments, high-risk populations for bladder cancer have characteristics including but not limited to smoking, work space exposure (e.g. makers of rubber, leather, textiles, painters, machinists, printers, hairdressers, or truck drivers), certain medicines or herbal supplements, arsenic in drinking water, not drinking enough fluids, race and ethnicity (Caucasians are about twice as likely to develop bladder cancer as African Americans and Hispanics, while Asians and American Indians have slightly lower rates of bladder cancer), age (about 9 out of 10 people with bladder cancer are older than 55), gender (bladder cancer is much more common in men than in women), chronic bladder irritation and infection, personal history of urothelial cancer or other bladder diseases, bladder birth defects, genetics and family history of bladder cancer, or chemotherapy or radiation therapy.

In some embodiments, patients are selected for cytopathological examination of urine samples on the basis that no other bladder cancer tissue is available for study, or invasive methods are not feasible.

In some embodiments, positive evaluation of bladder cancer based on the methods disclosed herein may drive a treatment regimen selection for the treatment of bladder cancer, such as chemotherapy, targeted therapy, and/or immunotherapy.

### EXAMPLES

Some aspects of the embodiments discussed herein are disclosed in further detail in the following examples, which are not in any way intended to limit the scope of the present disclosure. Those in the art will appreciate that many other embodiments also fall within the scope of the disclosure, as it is described herein and in the claims.

### Example 1. Multiple Staining of Urinary Sediment Cells by Immunohistochemistry And Morphological Stain

25 mL of a urine sample was fixed by mixing with an equal volume of CytoLyt (Hologic, Inc. MA, USA) or 4% paraformaldehyde. The sample was centrifuged to sediment the urinary cells. The supernatant was discarded and the urinary sediment cells were resuspended in 1 mL of PBS buffer (pH 7.4). 20 µL of Tween-20 was added and incubated for 5 minutes to permeabilize the cells, and the cells were subsequently isolated by centrifugation. 100 µL of Dual Endogenous Enzyme Block (Dako, Barpinteria, CA, USA) was added and incubated for 5 minutes to inhibit internal peroxidase and alkaline phosphatase activity, and the cells were subsequently isolated by centrifugation. 200 µL of Serum-Free Block (Dako) was added and incubated for 5 minutes. Then, 200 µL of primary rabbit anti-human cytokeratin 20 (CK-20) monoclonal antibody (1 µg/mL initial concentration; LBP Med Sci & Tech, Guangzhou, China) was added and incubated at room temperature for 2 hours. After primary antibody incubation, the cells were centrifuged and washed three times with 1 mL each of PBS buffer. 200 µL of goat anti-mouse secondary antibody conjugated with horseradish peroxidase (Envision, Dako) was added and incubated at room temperature for 1 hour. After secondary antibody incubation, the cells were centrifuged and washed three times with 1 mL each of PBS buffer. 100 µL Liquid DAB+ (Dako) was added and incubated for 2 minutes with agitation at 600 rpm. After chromogen incubation, the cells were centrifuged and washed three times with 1 mL each of PBS buffer. The final cell pellet was resuspended in 100 µL of PBS buffer pH 7.4. The resuspended cells were smeared onto glass slides, and Diff-Quik staining was performed with 10% Diff-Quik Solution (Fisher Scientific). The multiple stained cells were then evaluated under conventional microscopy.

### Centrifugation Steps Were Performed at 1500 Rpm for 5 Minutes

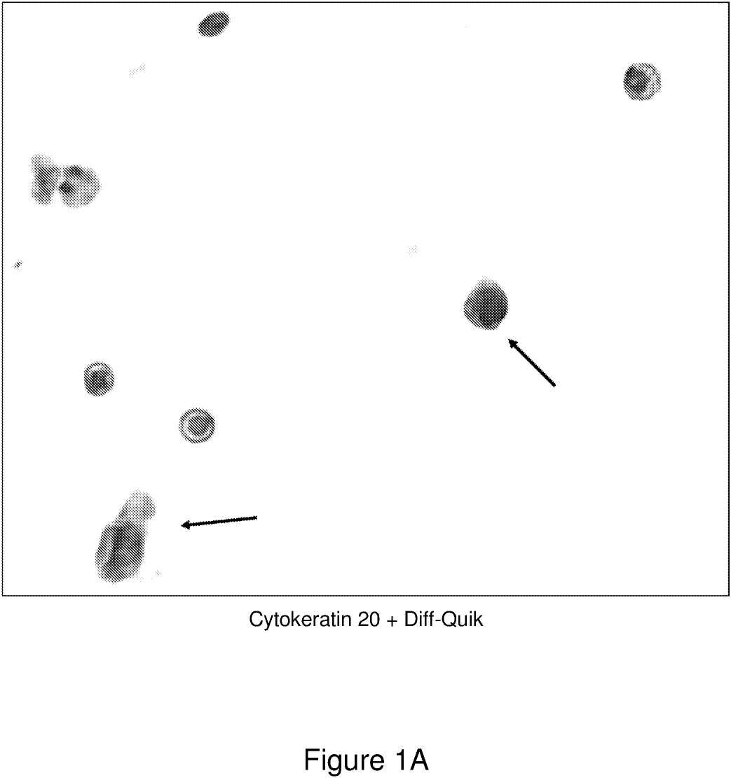

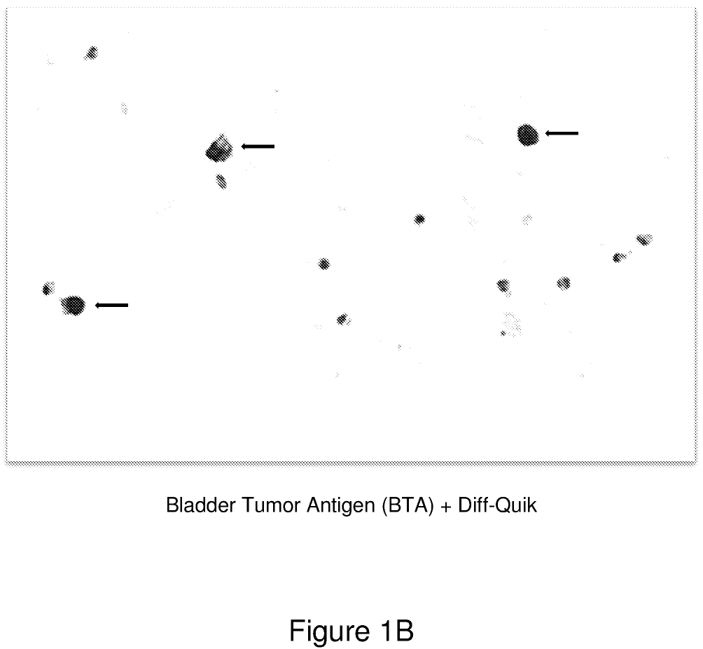

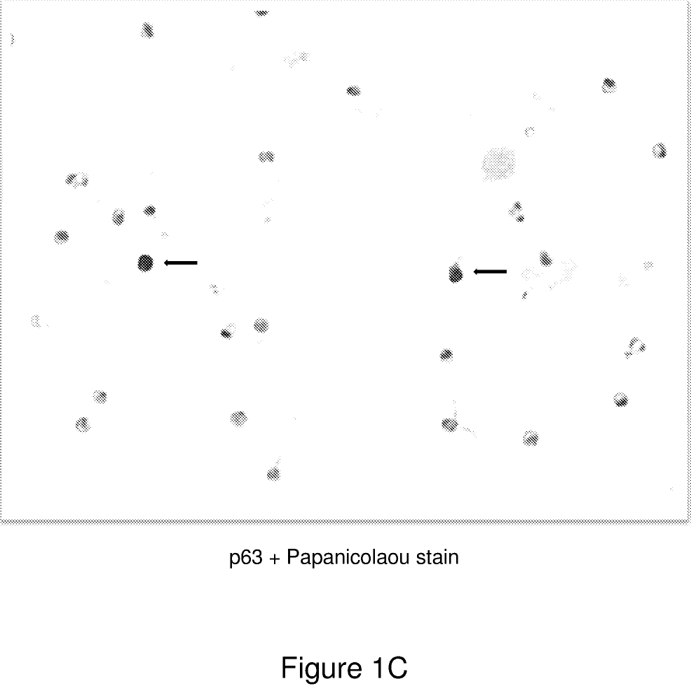

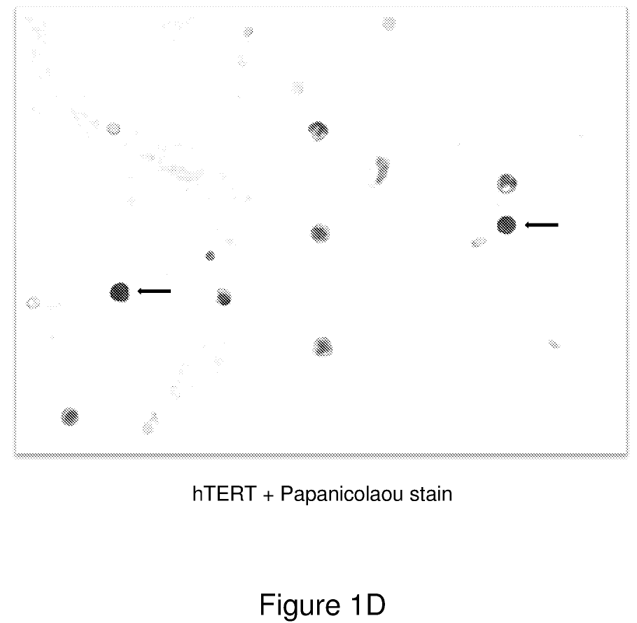

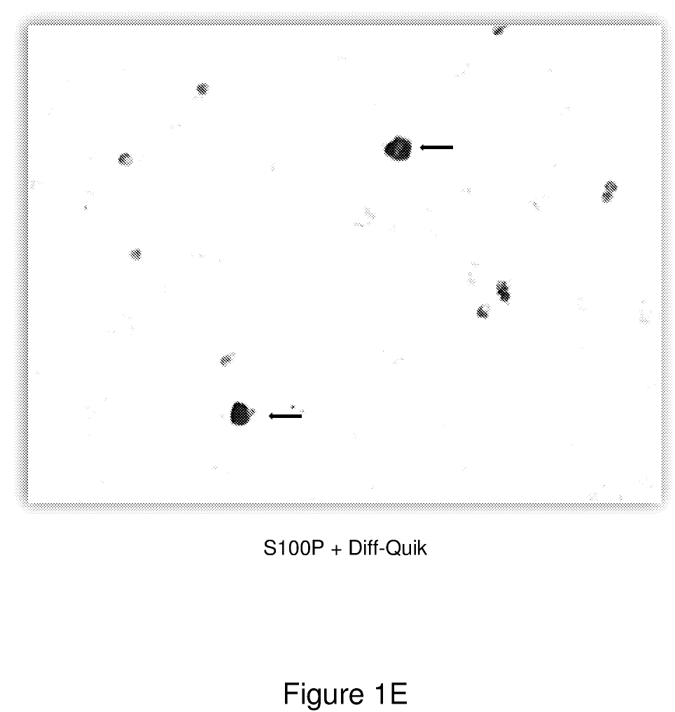

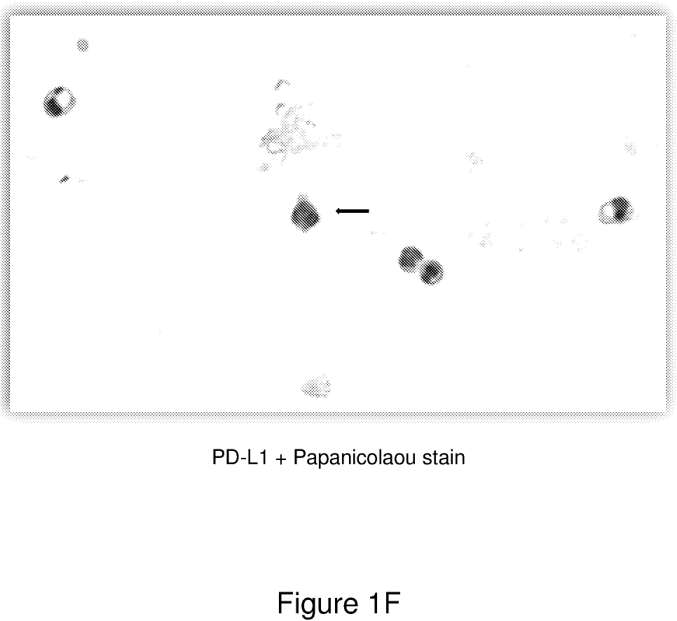

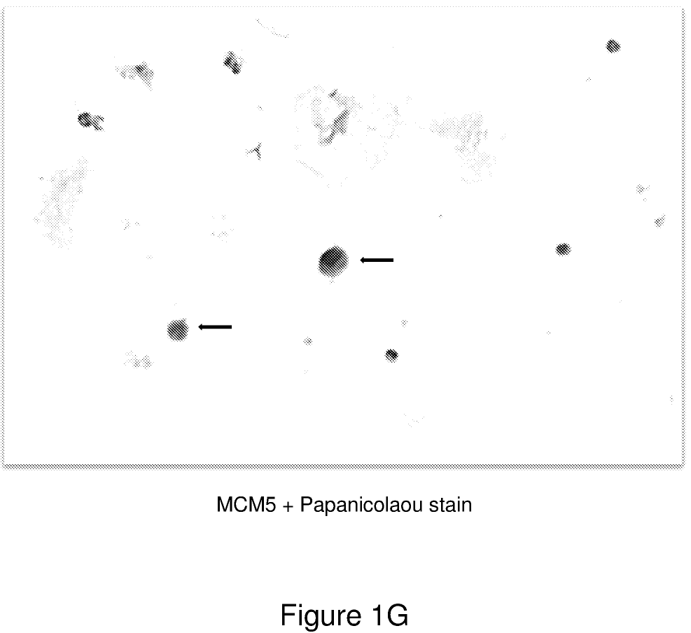

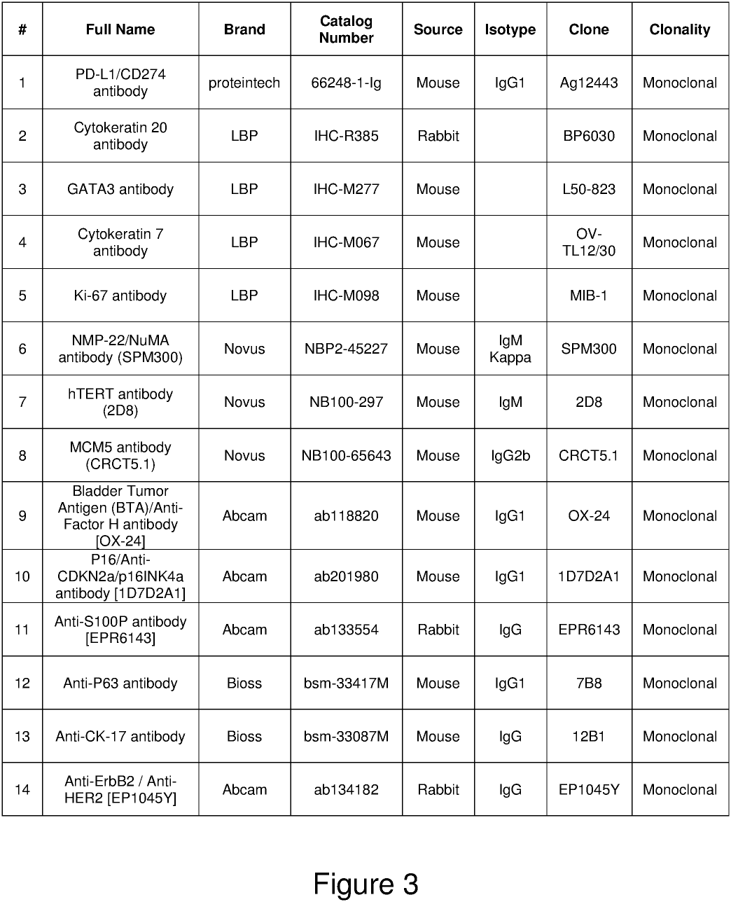

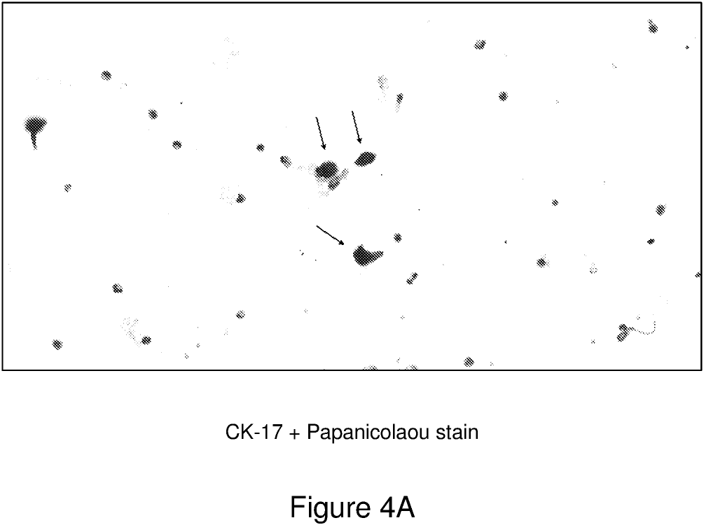

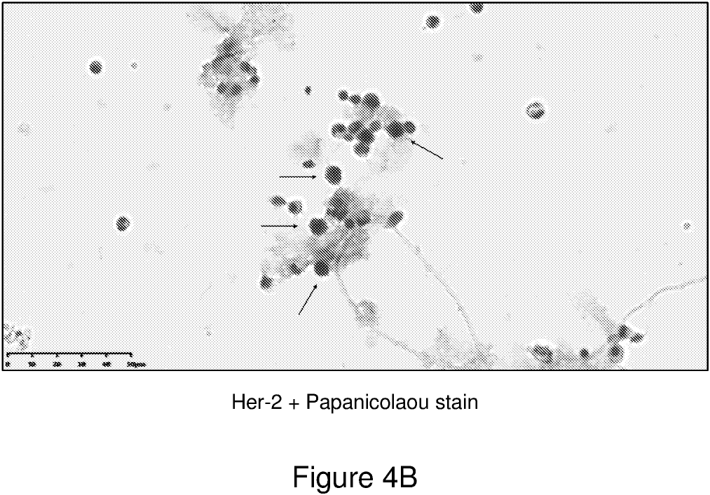

As seen in FIGS. 1A-G and 4A-B, this process results in urinary sediment cells doubly stained with 10% Diff-Quik or 5% Papanicolaou stain and immunocytochemistry. Atypical cells are easily observed as positive for the tested bladder cancer biomarker, while the morphological stain enables robust cytopathological identification. Biomarkers tested were human cytokeratin 20 (CK-20) (FIG. 1A), bladder tumor antigen (BTA) (FIG. 1B), p63 (FIG. 1C), hTERT (FIG. 1D), S100P (FIG. 1E), PD-L1 (FIG. 1F), and MCM5 (FIG. 1G). 5% Papanicolaou stain was substituted for 10% Diff-Quik for FIGS. 1C, 1D, 1F, and 1G to test staining with reduced methanol and/or ethanol concentrations. Additional biomarkers tested were cytokeratin-17 (CK-17) (FIG. 4A) and Her-2 (FIG. 4B) with Papanicolaou stain. Exemplary antibodies used to detect the biomarkers can be seen in FIG. 3. Each of FIGS. 1A-G and 4A-B were prepared using urine samples from different patients and therefore the cell morphology differ slightly between the images.

For each of the centrifugation steps for the staining procedure, a transwell procedure may be used instead. The transwell procedure involves the use of a suitably sized plate (e.g. 6 well plate) with a fitted insert, an insert or transwell, havinga porous membrane such that the aqueous reagents can pass through while the urinary sediment cells are retained. Optionally, a flange, protrusion, or handle is provided on the insert or transwell or plurality of inserts or transwells, e.g., connected to a rim or lip of the one or more inserts or transwells or a stem that connects a plurality of inserts or transwells, to facilitate moving one or more inserts or transwells from the wells. For example, a container, such as a plate, multi-well plate, or microtiter plate, e.g., having 1, 2, 4, 6, 8, 12, 24, 48, 64, 96 or 384 wells, and one or more inserts or transwells configured to fit into the well or wells of said container are used, wherein the one or more transwells comprises a porous membrane or screen that permits aqueous solution to pass but retains the cells of the cell sample, such as a porous membrane or screen having a pore size of 1, 2, 3, 4, 5, 6, 7, or 8 µm or having a pore size within a range defined by any two of the aforementioned pore sizes and, optionally wherein the one or more inserts or transwells further comprise a flange, protrusion, tab, or handle. This transwell procedure may be used instead of or in addition to a centrifuge and it is contemplated that the transwell procedure will preserve the cell morphology better and eliminate or reduce the amount of centrifugation. In some embodiments, centrifugation is desired for the initial sedimentation of the urinary exfoliated cells, especially when a high volume of biological sample having a low concentration of cells is used.

### Example 2. Multiple Staining of Urinary Sediment Cells by Chromogenic In-situ Hybridization And Morphological Stain

25 mL of a urine sample was fixed by mixing with an equal volume of CytoLyt or 4% paraformaldehyde. The sample was centrifuged to sediment the urinary cells. The supernatant was discarded and the urinary sediment cells were resuspended in 1 mL of PBS buffer pH 7.4. 20 µL of Tween-20 was added and incubated for 5 minutes to permeabilize the cells, and the cells were subsequently isolated by centrifugation. 200 µL of Hs-GATA3 probe (ACD Bio, CA, USA) was added and the mixture was incubated at 40° C. for 2 hours. 1 mL of washing buffer from the RNAscope 2.5 HD Reagent Kit (ACD Bio, CA, USA) was added, and the suspension was centrifuged. After discarding the supernatant, this washing step was repeated an additional time. 4 drops of hybridization solution AMP1 from the RNAscope 2.5 HD Reagent Kit was added and incubated at 40° C. for 30 minutes, then cells were washed twice with 1 mL of washing buffer and centrifuged. 4 drops of hybridization solution AMP2 from the RNAscope 2.5 HD Reagent Kit was added and incubated at 40° C. for 30 minutes, then cells were washed twice with 1 mL of washing buffer and centrifuged. 4 drops of hybridization solution AMP3 from the RNAscope 2.5 HD Reagent Kit was added and incubated at 40° C. for 30 minutes, then cells were washed twice with 1 mL of washing buffer and centrifuged. 4 drops of hybridization solution AMP4 from the RNAscope 2.5 HD Reagent Kit was added and incubated at 40° C. for 15 minutes, then cells were washed twice with 1 mL of washing buffer and centrifuged. 4 drops of hybridization solution AMP5 from the RNAscope 2.5 HD Reagent Kit was added and incubated at 40° C. for 30 minutes, then cells were washed twice with 1 mL of washing buffer and centrifuged. 4 drops of hybridization solution AMP6 from the RNAscope 2.5 HD Reagent Kit was added and incubated at 40° C. for 15 minutes, then cells were washed twice with 1 mL of washing buffer and centrifuged. 120 µL of mixed DAB-A and DAB-B from the RNAscope 2.5 HD Reagent Kit was added and incubated for 10 minutes with agitation at 600 rpm. After chromogen incubation, the cells were centrifuged and washed three times with 1 mL each of PBS buffer. The final cell pellet was resuspended in 100 µL of PBS buffer pH 7.4. The resuspended cells were smeared onto glass slides, and Diff-Quik staining was performed with 10% Diff-Quik solution (Fisher Scientific). The multiple stained cells were then evaluated under conventional microscopy. (Centrifugation steps were performed at 1500 rpm for 5 minutes).

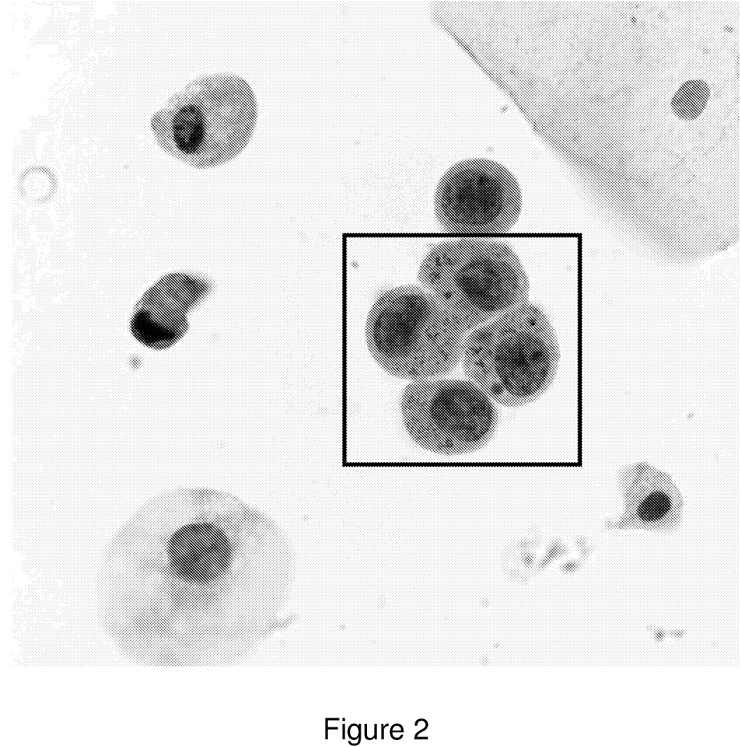

As seen in FIG. 2, this process results in urinary sediment cells doubly stained with Diff-Quik and GATA3 nucleic acid staining. Atypical cells are easily observed as positive for GATA3, while the morphological stain enables robust cytopathological identification.

For each of the centrifugation steps for the staining procedure, a transwell procedure may be used, as described in Example 1.

### Example 3. Clinical Trial Procedures

Patient enrollment: The clinical trial was approved by a local clinical trial ethics committee. All patients signed consent forms to participate in the clinical trial. All patients underwent cystoscopy, which currently is the conventional standard for initial bladder cancer diagnosis. Patients with bladder cancer (positive samples) were confirmed by cystoscopy and tissue biopsy. Patients without bladder cancer (negative samples) were confirmed by cystoscopy with or without tissue biopsy. 150 mL patient urine samples were collected from each patient, and the samples were fixed in an equal volume of Cytolyt (Hologic).

Staining performed: An admix of 20 mL patient urine sample and 20 mL Cytolyt were centrifuged, and urinary sediment cells were collected for each antibody stain. The sediment cells were stained with liquid-based immunocytochemistry as described herein, and concurrently stained by Papanicolaou stain for cytomorphological stain. Samples from each patient were separately stained with 4 monoclonal antibodies specific for CK-17, GATA-3, S100P, and hTERT. Other antibodies (or nucleic acid probes) as disclosed herein or otherwise known in the art as bladder cancer biomarkers can be used.

Bladder cancer cell enumeration: Immunohistochemically positive cells were counted for each antibody staining. The enumeration of positive cells was corrected by excluding false positive signals, such as non-specific staining on non-cellular debris in urinary samples. Furthermore, false positive staining on normal, non-cancerous cells was excluded. These non-cancerous cells include (1) normal squamous cells (which can be stained positively by CK-17, S100P, and hTERT), and (2) normal lymphocytes (which can be stained positively by GATA-3). False negatively stained cancer cells (under the limit of detection by antibodies) was included by cytomorphologic evaluation when possible. The enumeration correction was made possible by the cytomorphologic staining (e.g. Papanicolaou staining).

Final resulting: Based on the number of positive cancer cells from each of the antibody staining, a weighted score was calculated. The final scores will give rise to the following diagnoses:

a) Negative for bladder cancer: The final scores fall into the negative zone. There are not enough numbers for positively stained cells to support bladder cancer diagnosis.

b) Positive for bladder cancer: The final scores fall into the positive zone to render bladder cancer diagnosis.

c) Suspected bladder cancer: The final scores fall into a gray zone (rare situation).

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

With respect to the use of “e.g.”, it is understood to mean “for example” and is therefore a non-limiting example.

It will be understood by those within the art that, in general, terms used herein, and especially in the appended claims (e.g., bodies of the appended claims) are generally intended as “open” terms (e.g., the term “including” is typically interpreted as “including but not limited to,” the term “having” is typically interpreted as “having at least,” the term “includes” is typically interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases is typically construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” is typically interpreted to mean “at least one” or “one or more”); the same holds true for the use of definite articles used to introduce claim recitations. In addition, even if a specific number of an introduced claim recitation is explicitly recited, those skilled in the art will recognize that such recitation is typically interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “ a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, is typically understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference for any particular disclosure referenced herein and in their entirety, and are hereby made a part of this specification. To the extent publications and patents or patent applications incorporated by reference contradict the disclosure contained in the specification, the specification is intended to supersede and/or take precedence over any such contradictory material.

### References

Tabayoyong W & Kamat AM. Current Use and Promise of Urinary Markers for Urothelial Cancer. Curr Urol Rep. 2018;19(12):96.

Lopez-Beltran A, et al. Current and Emerging Bladder Cancer Biomarkers with an Emphasis on Urine Biomarkers. Expert Review of Molecular Diagnostics. 2020; 20(2): 231-243.

Zhu CZ et al. A review on the accuracy of bladder cancer detection methods. J Cancer. 2019;10(17):4038-4044.

Piaton E. et al. p16/Ki-67 Dual Labelling and Urinary Cytology Results According to the New Paris System for Reporting Urinary Cytology: Impact of Extended Follow-up. Cancer Cytopathol. 2017; 125(7):552-62.

Nataraju G & Gayathri MN, Compendium of Urinalysis-Urine Reagent Strips and Microscopy. Journal of medical science and clinical research; 2018. p. 6.

Becker GJ, et al. Advances in urine microscopy. American journal of kidney diseases. 2016;67:954-64.

Williams GJ, et al. Absolute and relative accuracy of rapid urine tests for urinary tract infection in children: a meta-analysis. The Lancet infectious diseases. 2010;10:240-50.

Kumar N, et al. Efficacy of voided urinary cytology and ultrasonography compared to cystoscopy in the detection of urinary bladder cancer. African journal of urology. 2017;23:192-6.

Lee P, et al. Sensitivity of Urine Cytology for High Grade Urothelial Carcinoma and Explanations for False-Negative Cytologies. Journal of the American society of cytopathology. 2015;6:S19-S20.

Anai S, et al. The sensitivity of the novel fluorescent urine cytology using 5-aminolevulinic acid (5-ALA) is higher than the conventional urine cytology. The journal of Urology; 2014. p. 191.

Hajdinjak T. UroVysion FISH test for detecting urothelial cancers: meta-analysis of diagnostic accuracy and comparison with urinary cytology testing. Urologic oncology: seminars and original investigations. 2008;26:646-51.

Babjuk M, et al. Urinary cytology and quantitative BTA and UBC tests in surveillance of patients with pTapT1 bladder urothelial carcinoma. Urology. 2008;71:718-22.

Tsui KH, et al. Comparisons of voided urine cytology, nuclear matrix protein-22 and bladder tumor associated antigen tests for bladder cancer of geriatric male patients in Taiwan, China. Asian journal of andrology. 2007;9:711-5.

Smith ZL & Guzzo TJ. Urinary markers for bladder cancer. F1000prime reports; 2013. p. 5.

Raitanen M-P & Group F. The role of BTA stat Test in follow-up of patients with bladder cancer: results from FinnBladder studies. World journal of urology. 2008;26:45-50.

Pajor G, et al. Urovysion: Considerations on modifying current evaluation scheme, including immunophenotypic targeting and locally set, statistically derived diagnostic criteria. Cytometry part A. 2011;79:375-82.

He H, et al. ImmunoCyt test compared to cytology in the diagnosis of bladder cancer: A meta-analysis. Oncology letters. 2016;12:83-8.

Comploj E, et al. uCyt+/ImmunoCyt and cytology in the detection of urothelial carcinoma. Cancer cytopathology. 2013;121:392-7.

Nguyen P-N, et al. A panel of TMPRSS2: ERG fusion transcript markers for urine-based prostate cancer detection with high specificity and sensitivity. European Urology. 2011;59:407-14.

Charpentier M, et al. Noninvasive Urine-Based Tests to Diagnose or Detect Recurrence of Bladder Cancer. Cancers. 2021;13(1650):1-23.

1. A method of detecting bladder cancer in a patient, comprising:
isolating urinary exfoliated cells from the patient;
fixing the urinary exfoliated cells in an aqueous cell suspension;
staining the urinary exfoliated cells with a liquid-based immunocytochemistry or chromogenic in-situ hybridization stain, or both;
staining the urinary exfoliated cells with a diagnostic cytomorphologic stain;
assessing the urinary exfoliated cells as comprising bladder cancer cells by the immunocytochemistry or chromogenic in-situ hybridization stain, or both, and the diagnostic cytomorphologic stain;
thereby detecting bladder cancer in the patient.

2-54. (canceled)

55. The method of claim 1, wherein the urinary exfoliated cells are urinary sediment cells isolated from the urine sample by centrifugation.

56. The method of claim 1, wherein the immunocytochemistry or chromogenic in-situ hybridization stain, or both, is performed in an aqueous cell suspension.

57. The method of claim 56, wherein the immunocytochemistry or chromogenic in-situ hybridization stain, or both, comprises labeling one or more bladder cancer specific biomarkers.

58. The method of claim 57, wherein the one or more bladder cancer specific biomarkers are selected from the group consisting of S100P, p63, M344, LDQ10, 19A211, GATA-3, Ki-67, p16, Her-2, PD-L1, CTLA4, CK-17, CK-20, nmp-22, bladder tumor antigen (BTA), hTERT, and mini-chromosome maintenance protein 5 (MCM5).

59. The method of claim 1, wherein the diagnostic cytomorphologic stain is performed either:
a) in an aqueous cell suspension; or
b) after mounting the urinary exfoliated cells onto a solid substrate.

60. The method of claim 59, wherein the diagnostic cytomorphologic stain comprises Diff-Quik stain, Papanicolaou stain, Wright-Giemsa stain, hematoxylin/eosin stain, or a derivative or modification thereof.

61. The method of claim 59, wherein the solid substrate is a microscope slide.

62. The method of claim 1, wherein the assessing step includes counting the numbers of total bladder cancer cells, or percentage of biomarker positive cells among bladder cancer cells, or both.

63. The method of claim 62, wherein the bladder cancer cells comprise one or more bladder cancer specific biomarkers selected from the group consisting of S100P, p63, M344, LDQ10, 19A211, GATA-3, Ki-67, p16, Her-2, PD-L1, CTLA4, CK-17, CK-20, nmp-22, bladder tumor antigen (BTA), hTERT, and mini-chromosome maintenance protein 5 (MCM5).

64. The method of claim 1, wherein the bladder cancer is urothelial carcinoma, squamous cell carcinoma, adenocarcinoma, small cell carcinoma, sarcoma, or any combination thereof.

65. The method of claim 1, further comprising treating the patient for bladder cancer.

66. The method of claim 65, wherein treating the patient for bladder cancer comprises surgery, chemotherapy, immunotherapy, targeted therapy, or radiation therapy, or any combination thereof.

67. The method of claim 66, wherein the chemotherapy comprises administration of cisplatin, carboplatin, fluorouracil, mitomycin, gemcitabine, methotrexate, vinblastine, doxorubicin, erdafitinib, afatinib, docetaxel, or paclitaxel, or any combination thereof.

68. The method of claim 66, wherein the immunotherapy comprises administration of Bacillus Calmette-Guerin (BCG), atezolizumab, avelumab, durvalumab, enfortumab, nivolumab, ipilimumab, trastuzumab, disitamab, PRS-343, or pembrolizumab, or any combination thereof.

69. The method of claim 65, wherein the patient is a human.

70. The method of claim 1, wherein the patient is selected to provide urinary exfoliated cells based on presentation of clinical symptoms of bladder cancer and/or being part of a population at high risk of developing bladder cancer.

71. The method of claim 1, wherein any one or more of the antibodies or binding fragments thereof set forth in Figure 3 are utilized.

72. The method of claim 1, wherein assessing the urinary exfoliated cells by the diagnostic cytomorphologic stain comprises assessing characteristics of the urinary exfoliated cells comprising nuclear features, shape or size of the nuclei or nucleoli; the density of chromatin; cytoplasmic elements such as mucins, fat droplets or neurosecretory granules; or cell membrane features such as membrane grooves, projections, or vacuoles.

73. The method of claim 1, wherein staining the urinary exfoliated cells with the liquid-based immunocytochemistry or chromogenic in-situ hybridization stain, and/or staining the urinary exfoliated cells with the diagnostic cytomorphologic stain comprises centrifugation or use of one or more inserts or transwells, wherein the one or more inserts or transwells comprise a porous membrane that permits aqueous solution to pass but retains the urinary exfoliated cells, such as a porous membrane having a pore size of 1, 2, 3, 4, 5, 6, 7, or 8 µm or having a pore size within a range defined by any two of the aforementioned pore sizes and, optionally, wherein the one or more inserts or transwells further comprise a flange, protrusion, tab, or handle.
